SEQUENCE LISTING (2 sequences)

SEQ ID NO: 1 - protein, 708 aa, Bos taurus
MKLFVPALLSLGALGLCLAAPRKNVRWCTISQPEWFKCRRWQWRMKKLGAPSITCVRRAFALECIRAIAEKKADAVTLDGGMVFEAGRDPYKLRPVAAEIYGTKESPQTHYYAVAVVKKGSNFQLDQLQGRKSCHTGLGRSAGWVIPMGILRPYLSWTESLEPLQGAVAKFFSASCVPCIDRQAYPNLCQLCKGEGENQCACSSREPYFGYSGAFKCLQDGAGDVAFVKETTVFENLPEKADRDQYELLCLNNSRAPVDAFKECHLAQVPSHAVVARSVDGKEDLIWKLLSKAQEKFGKNKSRSFQLFGSPPGQRDLLFKDSALGFLRIPSKVDSALYLGSRYLTTLKNLRETAEEVKARYTRVVWCAVGPEEQKKCQQWSQQSGQNVTCATASTTDDCIVLVLKGEADALNLDGGYIYTAGKCGLVPVLAENRKTSKYSSLDCVLRPTEGYLAVAVVKKANEGLTWNSLKDKKSCHTAVDRTAGWNIPMGLIVNQTGSCAFDEFFSQSCAPGRDPKSRLCALCAGDDQGLDKCVPNSKEKYYGYTGAFRCLAEDVGDVAFVKNDTVWENTNGESTADWAKNLNREDFRLLCLDGTRKPVTEAQSCHLAVAPNHAVVSRSDRAAHVKQVLLHQQALFGKNGKNCPDKFCLFKSETKNLLFNDNTECLAKLGGRPTYEEYLGTEYVTAIANLKKCSTSPLLEACAFLTR

SEQ ID NO: 2 - protein, 19 aa, Bos taurus
MKLFVPALLSLGALGLCLA

(19) United States
(12) Patent Application Publication
(10) Pub. No.: US 2024/0307507 A1
(43) Pub. Date: Sep. 19, 2024

(54) LACTOFERRIN COMPOSITIONS AND METHODS OF USE

(71) Applicant: LACTEA THERAPEUTICS LLC, Frederick, MD (US)

(72) Inventors: Christopher Hlubb, Ellicott City, MD (US); Jonathan Snedeker, Sykesville, MD (US)

(21) Appl. No.: 18/586,316

(22) Filed: Feb. 23, 2024

Related U.S. Application Data
(63) Continuation of application No. PCT/US2022/075367, filed on Aug. 23, 2022.
(60) Provisional application No. 63/236,204, filed on Aug. 23, 2021.
(60) Provisional application No. 63/304,434, filed on Jan. 28, 2022.

(51) Int. Cl. A61K 38/40; A61K 9/00; A61P 31/04; A61P 31/14

(52) U.S. Cl. CPC A61K 38/40; A61K 9/0043; A61K 9/0053; A61P 31/04; A61P 31/14

(57) ABSTRACT

Provided are compositions comprising Lactoferrin, in particular pharmaceutical formulations comprising Lactoferrin purified from raw natural milk sources. Also provided are methods of manufacturing and using the same.

### 1. CROSS REFERENCE TO RELATED APPLICATIONS

This application is a continuation of International Application No. PCT/US2022/075367, filed Aug. 23, 2022, which claims the benefit of U.S. Provisional Application No. 63/304,434, filed Jan. 28, 2022, and U.S. Provisional Application No. 63/236,204, filed Aug. 23, 2021, the entire contents of each is incorporated herein by reference for all purposes.

### 2. REFERENCE TO A SEQUENCE LISTING XML

The instant application contains a Sequence Listing which has been submitted electronically in XML format and is hereby incorporated by reference in its entirety. Said XML file, created on Aug. 30, 2022, is named HYA-001WO2_SL.xml, and is 3,495 bytes in size.

### 3. BACKGROUND

Lactoferrin has been explored in the context of its anti-microbial properties. However, preparations of lactoferrin are typically derived from previously processed and/or previously treated milk products, such as pasteurized milk sources, or recombinantly produced. Such production strategies can result in changes in the properties of Lactoferrin relative to that found in an unprocessed and/or untreated milk source, such as denaturation, reduced bioactivity, reduced iron binding capacity, altered glycosylation, and/or a lack of retention of post translational modifications. Additionally, available purified lactoferrin products generally contain significant impurities.

Absent from the field are preparations and formulations of lactoferrin directed to retaining lactoferrin's native bioactivity, including lactoferrin generally free of impurities (e.g., other proteins, enzymes, endotoxin, prions, etc.), including pharmaceutically acceptable (e.g., FDA registrable) preparations and formulations of lactoferrin, particular for use in the fields of wound care and other applications.

### 4. SUMMARY

Provided herein are pharmaceutical compositions comprising Lactoferrin. Also provided herein are compositions comprising Lactoferrin.

In some aspects, provided herein are pharmaceutical compositions comprising Lactoferrin, wherein the pharmaceutical composition is formulated for delivery to an oral cavity or nasal cavity. In some aspects, the pharmaceutical composition is formulated for retention of the Lactoferrin in the buccal mucosa. In some aspects, the pharmaceutical composition is formulated for delivery to the extra-cellular matrix (ECM) of a cell.

In some aspects, the pharmaceutical composition is formulated as a tablet. In some aspects, the tablet is formulated as an orally dissolvable or disintegrating tablet.

In some aspects, the pharmaceutical composition is formulated as a nasal spray.

In some aspects, the pharmaceutical composition is formulated as a gum.

In some aspects, the pharmaceutical composition is formulated as an oral thin film.

In some aspects, the pharmaceutical composition is formulated as a lozenge.

In some aspects, the oral cavity or nasal cavity comprises the nasopharynx.

Also provided herein is a pharmaceutical composition comprising Lactoferrin, wherein the pharmaceutical composition is formulated for delivery to an open wound or ulcer. In some aspects, the pharmaceutical composition is formulated for delivery to skin, mucosa, optic, connective, gastrointestinal and/or lung tissue. In some aspects, the wound comprises an open wound or ulcer. In some aspects, the ulcer is venous or arterial.

In some aspects, the wound is selected from the group consisting of: a burn, a bite, a cut, an infection, an ulceration, and combinations thereof. In some aspects, the burn is selected from the group consisting of: a thermal burn, a chemical burn, an electrical burn, a radiation burn, a friction burn, and combinations thereof. In some aspects, the cut is selected from the group consisting of: a puncture, a laceration, an abrasion, an incision, which may be surgical in nature, an amputation, and combinations thereof. In some aspects, the ulcer is venous or arterial.

In some aspects, the pharmaceutical composition comprises a bio-adhesive.

In some aspects, the pharmaceutical composition is formulated in a delivery format selected from the group consisting of: a gelfoam, a hydrogel, an infused bandage, a powder, a spray, and an inhalation format.

Also provided herein is a pharmaceutical composition comprising Lactoferrin, wherein the pharmaceutical composition is formulated for delivery to a gastrointestinal tract. In some aspects, the pharmaceutical composition is formulated in a delivery format comprising microencapsulation. In some aspects, wherein the pharmaceutical composition is formulated in a delivery format comprising an enteric coating.

Also provided herein is a composition comprising Lactoferrin, wherein the composition is formulated for delivery to an oral cavity, nasal cavity, wound, ocular cavity, and/or gastrointestinal tract. In some aspects, the wound comprises an open wound or ulcer. In some aspects, the ulcer is venous or arterial.

In some aspects, the pharmaceutical composition or composition comprises an excipient.

In some aspects, the pharmaceutical composition or composition comprises sodium steryl fumate, carbomer, sodium hydroxide, glycerin, sodium benzoate, benzoic acid, citric acid, and/or combinations thereof. In some aspects, the pharmaceutical composition or composition comprises xylitol, sorbitol, mannitol, maltitol, sugar alcohols, sucralose, and/or combinations thereof. In some aspects, the pharmaceutical composition or composition comprises Isomalt, microcrystalline cellulose, Sodium Carboxymethylcellulose, and/or combinations thereof. In some aspects, the pharmaceutical composition or composition comprises powdered sugar, gum base, corn syrup, gum Arabic, and/or combinations thereof. In some aspects, the pharmaceutical composition or composition comprises HiG PWD-04, Encapsulated powdered flavoring, Anhydrous or Natural Caffeine, Encapsulated Sucralose or Sucralose, Encapsulated Acesulfame-K and Aspartame, Aspartame, Acesulfame-K, Triacetin, Silicon dioxide, and/or combinations thereof.

In some aspects, the pharmaceutical composition or composition comprises a coagulant. In some aspects, the coagulant comprises thrombin, amylopectin, kaolin, and/or combinations thereof.

In some aspects, the pharmaceutical composition or composition comprises an astringent. In some aspects, the astringent is selected from the group consisting of: alum, acacia, sage, yarrow, witch hazel, bayberry, distilled vinegar, persimmon, green tea, black tea, citric acid, cranberry juice and/or extract, grapefruit juice and/or extract, inorganic acids, and combinations thereof.

In some aspects, the Lactoferrin is bovine. In some aspects, the Lactoferrin has not been treated. In some aspects, the Lactoferrin has not been chemically treated, enzymatically treated, acid treated, and/or heat treated. In some aspects, the Lactoferrin has not been heat treated. In some aspects, the Lactoferrin has not been heat treated at a temperature of 50° C. or greater, 51° C. or greater, 52° C. or greater, 53° C. or greater, 54° C. or greater, or 55° C. or greater. In some aspects, the Lactoferrin has not been heat treated at a temperature of 55° C. or greater.

In some aspects, the purified Lactoferrin comprises a native conformation as assessed by circular dichroism.

In some aspects, the purified Lactoferrin comprises a native conformation as assessed by Differential Scanning Calorimetry (DSC). In some aspects, the native conformation comprises an apolactoferrin and/or an hololactoferrin conformation. In some aspects, the apolactoferrin conformation has a melting temperature peak of 60.2+/−0.8° C. and/or the hololactoferrin conformation has a melting temperature peak of 88.38+/31 0.8° C.

In some aspects, the purified Lactoferrin is capable of binding iron. In some aspects, at least 30%, 40%, 50%, 60%, 70%, 80%, or 90% of the purified Lactoferrin is capable of binding iron. In some aspects, at least 91%, 92%, 93%, 94%, 95%, 96%, 97%, 98%, or 99% of the purified Lactoferrin is capable of binding iron. In some aspects, at least 99.1%, 99.2%, 99.3%, 99.4%, 99.5%, 99.6%, 99.7%, 99.8%, or 99.9% of the purified Lactoferrin is capable of binding iron. In some aspects, the capability to bind iron is assessed by DSC.

In some aspects, the purified Lactoferrin comprises a post-translational modification. In some aspects, the post-translational modification comprises glycosylation.

In some aspects, the purified Lactoferrin comprises an average molecular weight of at least 79000-86000 Da.

In some aspects, the purified Lactoferrin is dried. In some aspects, the purified Lactoferrin has been dried by freeze-drying/lyophilization, fluid-bed drying, or low-temperature spray-drying. In some aspects, the purified Lactoferrin remains in liquid form through Lactoferrin purification.

In some aspects, the composition further comprises an iron molecule. In some aspects, the purified Lactoferrin is complexed with an iron molecule. In some aspects, the iron molecule comprises Fe2+ or Fe3+. In some aspects, the purified Lactoferrin is complexed with a copper, zinc, manganese, and/or gallium molecule. In some aspects, the purified Lactoferrin is complexed with a zinc molecule.

In some aspects, the composition comprises endotoxin at a level of 5 EU/kg or less.

In some aspects, the Lactoferrin has been purified from a natural milk product that has not been treated. In some aspects, the natural milk product has not been processed prior to purification of the Lactoferrin. In some aspects, the natural milk product has not been chemically, enzymatically, acid, or heat treated prior to purification of the Lactoferrin. In some aspects, the natural milk product has not been heat treated prior to purification of the Lactoferrin. In some aspects, the heat treatment comprises a temperature of 50° C. or greater, 51° C. or greater, 52° C. or greater, 53°° C. or greater, 54° C. or greater, or 55°° C. or greater. In some aspects, the heat treatment comprises a temperature of 55°° C. or greater.

In some aspects, the Lactoferrin has been purified from a natural milk product that has been separated into skim milk and cream prior to purification of the Lactoferrin. In some aspects, the separation into skim milk and cream comprises cold-bowl separation. In some aspects, the Lactoferrin has been purified from a natural milk product has been acid treated prior to purification of the Lactoferrin. In some aspects, the acid treatment comprises removal of insoluble caseins. In some aspects, the acid treatment is at a pH of 4.0 or greater.

Also provided herein is a method of manufacturing any one of the pharmaceutical compositions or compositions provided herein. In some aspects, the method comprises one or more steps selected from the group consisting of: chromatography, filtration, and drying. In some aspects, the method comprises each of the steps of chromatography, filtration, and drying.

Also provided herein is a method of treating a disease or condition, the method comprising administering any one of the pharmaceutical compositions or compositions provided herein. In some aspects, the disease or condition is selected from the group consisting of: a pathogenic disease, a gastrointestinal (GI) infection, a wound infection, an allergic condition, an inflammatory condition, and combinations thereof.

Also provided herein is a method of treating a pathogenic disease, the method comprising administering any one of the above pharmaceutical compositions or compositions to a subject.

In some aspects, the administration is prophylactic. In some aspects, the subject is at risk for exposure to a pathogen. In some aspects, the subject has been exposed to a pathogen. In some aspects, the subject has been diagnosed with an infection by a pathogen.

Also provided herein is a method of reducing the risk of a pathogenic disease, the method comprising administering any one of the pharmaceutical compositions or compositions provided herein to a subject. In some aspects, the risk for a pathogenic disease comprises administration of the pharmaceutical compositions or compositions to a wound of the subject. In some aspects, the subject has been exposed to a pathogen and/or has been diagnosed with an infection by a pathogen.

In some aspects, pathogen comprises an orally and/or nasally transmitted microbe. In some aspects, the pathogen comprises an airborne microbe.

In some aspects, the pathogen comprises a virus. In some aspects, the virus comprises a coronavirus. In some aspects, the coronavirus is SARS-COV-2.

In some aspects, the pathogen comprises a bacterium.

In some aspects, the pathogen comprises a fungus.

In some aspects, treating the disease or condition comprises a prophylactic treatment of the disease or condition.

Also provide for herein is a method of reducing the risk of an infection, the method comprising administering any one of the above pharmaceutical compositions or compositions to a subject. In some aspects, the subject has a wound at risk for the infection.

Also provide for herein is a method of promoting wound healing of a tissue, the method comprising administering any one of the above pharmaceutical compositions or compositions to a wound of a subject's tissue. In some aspects, the wound is selected from the group consisting of: a burn, a cut, an infection, an ulceration, and combinations thereof. In some aspects, the burn is selected from the group consisting of: wherein the burn is selected from the group consisting of: a thermal burn, a chemical burn, an electrical burn, a radiation burn, a friction burn, and combinations thereof. In some aspects, the cut is selected from the group consisting of: a puncture, a laceration, an abrasion, an incision, an amputation, and combinations thereof. In some aspects, the tissue is skin, lung tissue, or combinations thereof. In some aspects, the method comprises treating a disease or condition selected from the group consisting of: a pathogenic disease, a gastrointestinal (GI) infection, a wound infection, an allergic condition, an inflammatory condition, and combinations thereof. In some aspects, treating the disease or condition comprises a prophylactic treatment of the disease or condition.

### 5. BRIEF DESCRIPTION OF THE SEVERAL VIEWS OF THE DRAWINGS

FIG. 1 illustrates a particular embodiment of a Lactoferrin purification process described herein. Briefly, raw milk (untreated milk, e.g., not chemically, enzymatically, acid, or heat treated prior to purification of the Lactoferrin) was (1) diverted prior to entry into an industry standard milk processing workflow; (2) flowed through an ion exchange resin/column with effluent typically (e.g., if requested/required by a raw milk producer) returned to the standard milk processing workflow and Lactoferrin-containing eluates collected; (3) collected eluates filtered; and (4) purified Lactoferrin processed.

FIG. 2A shows MALDI-TOF (˜18 kDa-100 kDa) purity assessment for Lactoferrin produced according to Example 1. The source material used was raw colostrum (RC).

FIG. 2B shows MALDI-TOF (˜18 kDa-100 kDa) purity assessment for Lactoferrin produced according to Example 1. The source material used was raw whole milk (RM).

FIG. 3A shows MALDI-TOF (˜18 kDa-100 kDa) purity assessment for an over the counter (OTC) Lactoferrin supplement.

FIG. 3B shows MALDI-TOF (˜18 kDa-100 kDa) purity assessment for a purchased laboratory reagent grade Lactoferrin.

FIG. 3C shows MALDI-TOF (˜18 kDa-100 kDa) purity assessment for a purchased laboratory reagent grade Lactoferrin.

FIG. 4 shows Orbitrap Velos mass-spectrometry purity assessment for Lactoferrin produced according to Example 1.

FIG. 5A shows an ELISA purity assessment for Lactoferrin produced according to Example 1 and a purchased laboratory reagent grade Lactoferrin. FIG. 5B shows Lactoferrin purity assessed by HPLC.

FIG. 6 shows Lactoferrin enrichment at the ECM by Immunofluorescent (IF) imaging.

FIG. 7 shows quantification of Lactoferrin enrichment at the ECM.

FIG. 8 shows Lactoferrin enrichment at the ECM by Immunofluorescent (IF) imaging in primary buccal cells.

FIG. 9 shows a Sars-COV-2 CPE Reduction assay in the presence of API-E2.

FIG. 10 shows a Differential Scanning Calorimetry (DSC) assay plot assessing API-E2 Lactoferrin (10 mg/mL), including a heat-treated version.

FIG. 11A shows a DSC assay plot assessing API-E2 Lactoferrin in the absence and presence of excess iron.

FIG. 11B shows a DSC assay plot assessing Lab-grade standard lactoferrin and a commercially available supplement-grade lactoferrin overlaid on top of a plot of API-E2 Lactoferrin (see FIG. 10).

FIG. 12 shows an assessment of lactoferrin bioactivity on salivary bacteria as an assessment of pH over time in the presence of API-E2.

FIG. 13 shows pictures of poultry untreated (left) or treated with API-E2 (right) inoculated with salivary human bacteria and left for 6 days at 37° C.

FIG. 14 shows a zone of inhibition test was performed to assess bioactivity of API-E2 Lactoferrin for E. coli.

FIG. 15 shows a lactoperoxidase (LPO) activity outline and results of various lactoferrin samples, including API-E2.

### 6. DETAILED DESCRIPTION

### Definitions

Terms used in the claims and specification are defined as set forth below unless otherwise specified.

“Unprocessed milk product” or “unprocessed Lactoferrin-comprising milk product” as used interchangeably herein refers to the natural liquid lactation product produced by the mammary glands of a mammal and collected directly from the mammal to which no additional processing (e.g., filtration, column/resin purification, and/or milk separation) and/or treatment steps have been applied (e.g., chemical treatment, enzymatic treatment, acid treatment, and/or heat treatment).

“Untreated milk product” or “untreated Lactoferrin-comprising milk product” as used interchangeably herein refers to a milk product that has not undergone a treatment step (e.g., chemical treatment, enzymatic treatment, acid treatment, and/or heat treatment) but is inclusive of having undergone one or more potential mechanical processing steps (e.g., filtration, column/resin purification, and/or milk separation).

Unless specifically stated or otherwise apparent from context, as used herein the term “about” is understood as within a range of normal tolerance in the art, for example within 2 standard deviations of the mean. About can be understood as within 10%, 9%, 8%, 7%, 6%, 5%, 4%, 3%, 2%, 1%, 0.5%, 0.1%, 0.05%, or 0.01% of the stated value. Numerical values provided herein can sometimes be considered to be modified by the term about, where context makes clear that the ranges encompassed by the modification are consistent with operability of the invention and definiteness of the claims.

Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by visual inspection (see generally Ausubel et al.).

One example of an algorithm that is suitable for determining percent sequence identity and sequence similarity is the BLAST algorithm, which is described in Altschul et al., J. Mol. Biol. 215:403-410 (1990). Software for performing BLAST analyses is publicly available through the National Center for Biotechnology Information.

### Lactoferrin

Compositions that include purified Lactoferrin are provided herein. In general, Lactoferrin herein refers to a purified form of mammalian Lactoferrin obtained from an unprocessed milk product (e.g., raw milk).

The Lactoferrin of the purified compositions herein is typically bovine, e.g., purified from a bovine milk source. Exemplary bovine Lactoferrin (bLF) molecules include, but are not limited to, CAS Reg. No. 146897-68-9 and those described in Mead and Tweedie (Nucleic Acids Res. 1990 Dec. 11; 18(23):7167.) and Pierce et al. (Eur J Biochem. 1991 Feb. 26; 196(1):177-84.), each of which is incorporated herein by reference for all purposes. An exemplary non-limiting full-length amino acid sequence of bovine (Bos taurus) Lactoferrin is provided by the following:

MKLFVPALLSLGALGLCLAAPRKNVRWCTISQPEWFKCRRWQWRMKKLG
APSITCVRRAFALECIRAIAEKKADAVTLDGGMVFEAGRDPYKLRPVAA
EIYGTKESPQTHYYAVAVVKKGSNFQLDQLQGRKSCHTGLGRSAGWVIP
MGILRPYLSWTESLEPLQGAVAKFFSASCVPCIDRQAYPNLCQLCKGEG
ENQCACSSREPYFGYSGAFKCLQDGAGDVAFVKETTVFENLPEKADRDQ
YELLCLNNSRAPVDAFKECHLAQVPSHAVVARSVDGKEDLIWKLLSKAQ
EKFGKNKSRSFQLFGSPPGQRDLLFKDSALGFLRIPSKVDSALYLGSRY
LTTLKNLRETAEEVKARYTRVVWCAVGPEEQKKCQQWSQQSGQNVTCAT
ASTTDDCIVLVLKGEADALNLDGGYIYTAGKCGLVPVLAENRKTSKYSS
LDCVLRPTEGYLAVAVVKKANEGLTWNSLKDKKSCHTAVDRTAGWNIPM
GLIVNQTGSCAFDEFFSQSCAPGRDPKSRLCALCAGDDQGLDKCVPNSK
EKYYGYTGAFRCLAEDVGDVAFVKNDTVWENTNGESTADWAKNLNREDF
RLLCLDGTRKPVTEAQSCHLAVAPNHAVVSRSDRAAHVKQVLLHQQALF
GKNGKNCPDKFCLFKSETKNLLFNDNTECLAKLGGRPTYEEYLGTEYVT
AIANLKKCSTSPLLEACAFLTR (SEQ ID NO: 1; GenBank
Accession number AAA30610.1).

Lactoferrin can be a fragment of full-length Lactoferrin (e.g., SEQ ID NO:1). Fragments include biologically active fragments. As used herein, “biologically active” refers to a protein having one or more of the bioactivities of a corresponding native protein, including but not limited to enzymatic activity, anti-microbial activity (e.g., anti-bacterial, anti-fungal, and/or anti-viral activity), iron-binding/sequestration activity, immunomodulatory behavior (e.g., anti-inflammatory activity), growth regulation, and cell-surface affinity, wound healing, or any of the other activities of Lactoferrin described herein or known in the art. For example, Lactoferrin is typically secreted and biologically active fragments can include secreted formats, such as “processed” fragments of Lactoferrin lacking a signal peptide. As an illustrative example, the Lactoferrin represented by SEQ ID NO: 1 includes the signal peptide MKLFVPALLSLGALGLCLA (SEQ ID NO:2; amino acids 1-19 of SEQ ID NO:1). Accordingly, biologically active fragments of Lactoferrin can include Lactoferrin lacking a signal peptide (e.g., amino acids 20-708 of SEQ ID NO:1).

Lactoferrin can be a lactoferrin isoform, such as a Lactoferrin alpha (LFα), Lactoferrin beta (LFβ), or Lactoferrin gamma (LFγ) isoform.

Lactoferrin can have an amino acid sequence at least 75%, 80%, 81%, 82%, 83%, 84%, 85%, 86%, 87%, 88%, 89%, 90%, 91%, 92%, 93%, 94%, 95%, 96%, 97%, 98%, or 99% identical to an amino acid sequence set forth as SEQ ID NO: 1 or biologically active fragments thereof (e.g., a secreted form, such as amino acids 20-708 of SEQ ID NO: 1 lacking a signal peptide). Lactoferrin can have an amino acid sequence at least 95% identical to an amino acid sequence set forth as SEQ ID NO: 1 or biologically active fragments thereof. Lactoferrin can have an amino acid sequence at least 96% identical to an amino acid sequence set forth as SEQ ID NO: 1 or biologically active fragments thereof. Lactoferrin can have an amino acid sequence at least 97% identical to an amino acid sequence set forth as SEQ ID NO: 1 or biologically active fragments thereof. Lactoferrin can have an amino acid sequence at least 98% identical to an amino acid sequence set forth as SEQ ID NO: 1 or biologically active fragments thereof. Lactoferrin can have an amino acid sequence at least 99% identical to an amino acid sequence set forth as SEQ ID NO: 1 or biologically active fragments thereof. Lactoferrin can have an amino acid sequence at least 99.5% identical to an amino acid sequence set forth as SEQ ID NO: 1 or biologically active fragments thereof.

Lactoferrin can have a conservative substitution. A “conservative substitution” or a “conservative amino acid substitution,” refers to the substitution an amino acid with a chemically or functionally similar amino acid. Conservative substitutions are well known in the art, for example, as described in Creighton, Proteins: Structures and Molecular Properties 2nd ed. (1993) W. H. Freeman & Co., New York, NY, herein incorporated by reference for all purposes.

### Post-Translational Modifications

Lactoferrin found in an unprocessed milk product as a source material typically includes post-translational modifications. Without wishing to be bound by theory, the purification strategies and methods described herein are designed to reduce, minimize, or eliminate disrupting native post-translational modifications with reference to those found in the natural milk source used for Lactoferrin purification. For example, the purification strategies and methods described herein reduce, minimize, or eliminate chemical treatment, enzymatic treatment, acid treatment, heat treatment (e.g., pasteurization), and/or any other treatment capable of disrupting native post-translational modifications.

Post-translational modifications can be those modifications involved in Lactoferrin's bioactivity. In general, recombinantly-produced Lactoferrin, which is typically produced in an exogenous expression system such as bacteria or yeast, lacks post-translational modifications and/or post-translational modifications of natively produced Lactoferrin. Post-translational modifications include, but are not limited to, glycosylation, phosphorylation, and acetylation. Post-translational modifications in particular can include glycosylation (e.g., N-linked glycosylation), such as glycosylation at asparagine 233, 281, 368, 476, and/or 545, which can include-acetylneuraminic acid, galactose, mannose, fucose, N-acetylglucosamine, and/or N-acetylgalactosamine. Post-translational modifications can be naturally occurring and/or non-naturally occurring (e.g., modified following purification, such as by in vitro methods known to those skilled in the art). Post-translational modifications include processing of full-length Lactoferrin, such as removal of the signal sequence, as described above.

Unmodified secreted Lactoferrin typically has a molecular weight of about 78 kDa. Native post-translation modifications can result in a molecular weight ranging up to about 86 kDa. The purified Lactoferrin can have a molecular weight of greater than about 78 kDa. The purified Lactoferrin can have a molecular weight of at least 79 kDa. The purified Lactoferrin can have a molecular weight of at least 80 kDa. The purified Lactoferrin can have a molecular weight of at least 81 kDa. The purified Lactoferrin can have a molecular weight of at least 82 kDa. The purified Lactoferrin can have a molecular weight of at least 83 kDa. The purified Lactoferrin can have a molecular weight of at least 84 kDa. The purified Lactoferrin can have a molecular weight of at least 85 kDa. The purified Lactoferrin can have a molecular weight of at least 86 kDa. The purified Lactoferrin can have a molecular weight of between 79-86 kDa. The purified Lactoferrin can have a molecular weight of between 82-84 kDa. The purified Lactoferrin can have a molecular weight of between 82-85 kDa.

Methods of assessing post-translational modifications are known to those of skill in the art, such as mass-spectrometry or antibody-mediated methods (e.g., use of antibodies that recognize post-translational modifications, such as ELISA, and/or two-dimensional Western blot analysis).

### Protein Conformation

The conformational state of Lactoferrin found in an unprocessed milk product is typically considered the native conformation of Lactoferrin. Various treatments of milk sources typically used for purification of Lactoferrin, such as pasteurization, are generally considered capable of denaturing proteins. Without wishing to be bound by theory, the purification strategies and methods described herein are designed to reduce, minimize, or eliminate disrupting a native conformational state with reference to conformational states found in the natural milk source used for Lactoferrin purification. For example, the purification strategies and methods described herein reduce, minimize, or eliminate chemical treatment, enzymatic treatment, acid treatment, heat treatment (e.g., pasteurization), and/or any other treatment capable of denaturing Lactoferrin.

Methods of assessing the conformational state are known to those of skill in the art, such as circular-dichroism, x-ray crystallography, or antibody-mediated methods (e.g., use of antibodies that recognize conformational state and/or non-denaturing Western blot analysis).

### Iron Complexing

Lactoferrin includes two iron-binding domains (also referred to as globular lobes). Without wishing to be bound by theory, iron-binding properties can mediate and/or influence anti-microbial bioactivities, such as microbial killing, prevention of microbial entry, chelation, microbial elimination, and/or inhibition of growth.

Iron-bound Lactoferrin is referred to as hololactoferrin and iron-free Lactoferrin referred to as apolactoferrin and can, in some instances, differ in bioactivity, such as their antibacterial activity, or other properties, such as hololactoferrin's increased resistance to heat induced changes relative to apolactoferrin. Lactoferrin in an unprocessed milk product typically is found within a defined ratio of iron-free to iron-bound forms. For example, Lactoferrin in bovine milk generally has 20-30% of Lactoferrin present in the iron-bound form and in human milk generally has 6-8% present in the iron-bound form. Purified lactoferrin in compositions herein can have a defined range of iron-bound hololactoferrin. Purified lactoferrin can be between 20-30% hololactoferrin. Purified lactoferrin can be between 6-8% hololactoferrin. Purified lactoferrin can be greater than 30% hololactoferrin. Purified lactoferrin can be less than 6% hololactoferrin. Purified lactoferrin can be at least 30%, 40%, 50%, 60%, 70%, 80%, or 90% hololactoferrin. Purified lactoferrin can be at least 91%, 92%, 93%, 94%, 95%, 96%, 97%, 98%, or 99% hololactoferrin. Purified lactoferrin can be at least 99.1%, 99.2%, 99.3%, 99.4%, 99.5%, 99.6%, 99.7%, 99.8%, or 99.9% hololactoferrin. Purified lactoferrin can be 100% hololactoferrin. Purified lactoferrin can be less than 20% hololactoferrin. Purified lactoferrin can be greater than 8% hololactoferrin. Purified lactoferrin can be less than 1%, 2%, 3%, 4%, 5%, 6%, 7%, 8%, or 9% hololactoferrin. Purified lactoferrin can be less than 11%, 12%, 13%, 14%, 15%, 16%, 17%, 18%, or 19% hololactoferrin. Purified lactoferrin can be 100% in the iron-free apolactoferrin form.

Differential Scanning Calorimetry (DSC) can be used to assess the capability of purified lactoferrin to be in its hololactoferrin form, e.g., assess the capability of purified lactoferrin to bind iron. For example, the ability to bind iron can be assessed by adding excess iron in the presence of purified lactoferrin then performing DSC to assess the relative peaks associated with apolactoferrin and hololactoferrin. Purified lactoferrin and/or a dosage formula can include purified lactoferrin where at least 30%, 40%, 50%, 60%, 70%, 80%, or 90% of the purified Lactoferrin is capable of binding iron. Purified lactoferrin and/or a dosage formula can include purified lactoferrin where at least 30%, 40%, 50%, 60%, 70%, 80%, or 90% of the purified Lactoferrin is capable of binding iron, as assessed by DSC. Purified lactoferrin and/or a dosage formula can include purified lactoferrin where at least 91%, 92%, 93%, 94%, 95%, 96%, 97%, 98%, or 99% of the purified Lactoferrin is capable of binding iron. Purified lactoferrin and/or a dosage formula can include purified lactoferrin where at least 91%, 92%, 93%, 94%, 95%, 96%, 97%, 98%, or 99% of the purified Lactoferrin is capable of binding iron, as assessed by DSC. Purified lactoferrin and/or a dosage formula can include purified lactoferrin where at least 99.1%, 99.2%, 99.3%, 99.4%, 99.5%, 99.6%, 99.7%, 99.8%, or 99.9% of the purified Lactoferrin is capable of binding iron. Purified lactoferrin and/or a dosage formula can include purified lactoferrin where at least 99.1%, 99.2%, 99.3%, 99.4%, 99.5%, 99.6%, 99.7%, 99.8%, or 99.9% of the purified Lactoferrin is capable of binding iron, as assessed by DSC. Purified lactoferrin and/or a dosage formula can include purified lactoferrin where 100% of the purified Lactoferrin is capable of binding iron. Purified lactoferrin and/or a dosage formula can include purified lactoferrin where 100% of the purified Lactoferrin is capable of binding iron, as assessed by DSC (e.g., the only observable peak in the presence of excess iron is the hololactoferrin-associated peak).

Differential Scanning Calorimetry (DSC) can be used to assess melting temperature peaks of purified lactoferrin, in particular melting temperature peaks of the apolactoferrin and/or hololactoferrin. Purified lactoferrin and/or a dosage formula can include purified lactoferrin where a melting temperature peak associated with apolactoferrin is 60.2+/−0.8° C. Purified lactoferrin and/or a dosage formula can include purified lactoferrin where a melting temperature peak associated with hololactoferrin is 88.38+/−0.8° C.

Lactoferrin in the hololactoferrin form typically binds two ferric (Fe³⁺) ions in natural milk sources. Purified lactoferrin in compositions herein can be bound to metal ions other than ferric ions including, but not limited to copper, zinc, manganese, and/or gallium. Purified lactoferrin can be bound to zinc ions. Purified lactoferrin can be bound to Fe²⁺ions. Purified lactoferrin bound to metal ions other than ferric ions can be in any of the hololactoferrin or apolactoferrin forms of ferric-bound Lactoferrin described herein, e.g., any of the defined ratios of hololactoferrin to apolactoferrin forms described herein.

Conformational states of Lactoferrin, e.g., hololactoferrin and apolactoferrin conformations, can be saturated with at various level of metal ions (e.g., Fe³⁺). For example, apolactoferrin typically is saturated with less than 5% iron ions while hololactoferrin is typically about 100% saturation. Bovine Lactoferrin in an unprocessed milk product typically is 15-20% iron saturated.

Methods of controlling the defined ratios of hololactoferrin to apolactoferrin forms are known to those of skill in the art. For example, methods of controlling the concentration of ferric ions, metal ions other than ferric ions, and/or non-ferric iron-based and/or iron-derived molecules are known in the art, such addition or removal (e.g., as described in Majka et al. [Analytical and Bioanalytical Chemistry volume 405, pages 5191-5200 (2013)], herein incorporated by reference for all purposes).

Methods of assessing the ability of purified Lactoferrin to bind metal ions are known to those of skill in the art, such as chemical assays and/or absorbance spectroscopy. For example, without wishing to be bound by theory, treatment processes (e.g., those typically used in industrial purification) and/or recombinant production processes can alter the metal-binding ability of Lactoferrin (e.g., through denaturation of iron-binding domains).

Methods of assessing a ratio of hololactoferrin to apolactoferrin forms are known to those of skill in the art, such as chemical assays and/or absorbance spectroscopy (e.g., as described in Majka et al. [Analytical and Bioanalytical Chemistry volume 405, pages 5191-5200 (2013)], herein incorporated by reference for all purposes).

In a non-limiting example, Differential Scanning Calorimetry (DSC) provides both a method of assessing the ability of purified Lactoferrin to bind metal ions and assessing a ratio of hololactoferrin to apolactoferrin forms. DSC methods are known to those of skill in the art.

### Milk Product Purification

Purification methods can reduce, minimize, or eliminate chemical treatment, enzymatic treatment, acid treatment, and/or heat treatment. Purification methods can reduce chemical treatment, enzymatic treatment, acid treatment, and/or heat treatment. Purification methods can minimize chemical treatment, enzymatic treatment, acid treatment, and/or heat treatment. Purification methods can eliminate chemical treatment, enzymatic treatment, acid treatment, and/or heat treatment. Purification methods can reduce, minimize, or eliminate chemical treatment. Purification methods can reduce, minimize, or eliminate enzymatic treatment. Purification methods can reduce, minimize, or eliminate acid treatment. Purification methods can reduce, minimize, or eliminate heat treatment. Purification methods can reduce each of a chemical treatment, enzymatic treatment, acid treatment, and heat treatment. Purification methods can minimize each of a chemical treatment, enzymatic treatment, acid treatment, and heat treatment. Purification methods can eliminate each of a chemical treatment, enzymatic treatment, acid treatment, and heat treatment. Purification methods can eliminate each of a chemical treatment, enzymatic treatment, and acid treatment. Purification methods can eliminate each of a chemical treatment, enzymatic treatment, and heat treatment.

In general, methods provided herein for the production of purified Lactoferrin do not include heat treatments typical in industrial purification processes (e.g., pasteurization) that can disrupt a native conformational state (e.g., denaturing) with reference to conformational states found in the natural milk source used for Lactoferrin purification. Typical heat treatments used in industrial purification processes can be about 63° C. or greater. Heat treatments can be 50° C. or greater, 51° C. or greater, 52° C. or greater, 53° C. or greater, 54° C. or greater, or 55° C. or greater. Heat treatments can be 70°° C. or greater, 75° C. or greater, 80° C. or greater, 85° C. or greater, 90° C. or greater, 95° C. or greater, or 100° C. or greater. Purification methods can include performing processes at temperatures that reduce, minimize, or eliminate disrupting a native conformational state with reference to conformational states found in the natural milk source used for Lactoferrin purification relative to heat treatments typical in industrial purification processes. Purification methods can include performing processes at temperatures that reduce, minimize, or eliminate a reduction in Lactoferrin bioactivity with reference to bioactivity found in the natural milk source used for Lactoferrin purification relative to heat treatments typical in industrial purification processes. Purification methods can include receiving the natural milk source at refrigerated temperatures (e.g., at a temperature of less than 15° C., such as within a temperature between 2-15° C.). Purification methods described herein can include heat treatments of less than 50° C. For example, milk sources can be warmed during one or more purification steps, e.g., warmed to a temperature above 37°° C., but not to exceed 55° C. Warming can include a temperature above 37° C., 38° C., 39° C., 40° C., 41° C., 42° C., 43° C., 44° C., 45°° C., 46° C., 47° C., 48° C., 49° C., 50° C., 51° C., 52° C., 53° C., 54° C., or 55° C. Warming can include a temperature above 37° C. and at a temperature of less than 55° C. Warming can include a temperature above 40° C. and at a temperature of less than 55° C. Warming can include a temperature above 45° C. and at a temperature of less than 55° C. Warming can include a temperature above 50° C. and at a temperature of less than 55° C. Warming can include a temperature above 37° C. and at a temperature of 50° C. or less, 51° C. or less, 52° C. or less, 53° C. or less, 54° C. or less, or 55° C. or less. Warming can include a temperature above 40° C. and at a temperature of 50° C. or less, 51° C. or less, 52° C. or less, 53° C. or less, 54° C. or less, or 55° C. or less. Warming can include a temperature above 45° C. and at a temperature of 50° C. or less, 51° C. or less, 52° C. or less, 53° C. or less, 54° C. or less, or 55° C. or less. Purification methods described herein can include heat treatments of 50° C. or less, 51° C. or less, 52° C. or less, 53° C. or less, 54° C. or less, or 55° C. or less. Purification methods described herein can include maintaining a temperature during purification below 50° C. or less, 51° C. or less, 52° C. or less, 53° C. or less, 54° C. or less, or 55° C. or less. Maintaining a temperature during purification can include maintaining a temperature throughout purification. Maintaining a temperature during purification can include maintaining a temperature in one or more individual steps of a purification process (e.g., chromatography, filtration, and/or drying steps). A maintained temperature can include varying temperatures (e.g., differing temperature ranges) specific to one or more individual steps of a purification process.

Purification methods can include acid treatments. Without wishing to be bound by theory, acid treatments can be used for removal of caseins, such as treating a natural milk source or derivative prior to Lactoferrin purification at a pH capable of causing caseins to become insoluble in solution. In general, acid treatments provided herein for the production of purified Lactoferrin do not include acids treatments that disrupt a native conformational state (e.g., denature Lactoferrin) and/or reduce Lactoferrin bioactivity with reference to conformational states or bioactivity, respectively, found in the natural milk source used for Lactoferrin purification. Purification methods can include acid treatments at a pH of 4.0 or greater. Purification methods can include acid treatments at a pH of 3.0 or greater.

Purification methods can include chromatography. Purification methods can include ion exchange chromatography. Methods of chromatography, such as ion exchange chromatography, are known to those of skill in the art. The purification methods described herein will generally include cation exchange chromatography. Purification methods may include ion exchange chromatography steps in addition to cation exchange chromatography, such as both cation and anion exchange chromatography, including in any order and/or separated be one or more addition purification processes. Resins and matrices for ion exchange chromatography are known in the art. For example, cation exchange resins include, but are not limited to, polymethacrylate and agarose matrices. Purification methods can include high-pressure liquid chromatography (HPLC).

Chromatography methods in general include one or more equilibration and/or regeneration steps. An illustrative non-limiting example of equilibration and regeneration steps includes rinsing with (1) reverse osmosis water; (2) chemically pure 1M NaCl; and (3) rinsing again with reverse osmosis water.

Chromatography methods in general include a loading step. In general, the volume loaded is based on a pre-determined binding capacity of a resin and an estimation of the native Lactoferrin content of a raw milk feed material.

Chromatography methods in general include one or more elution steps, e.g., elution of purified Lactoferrin off a resin/column in ion exchange chromatography. Methods of elution are known to those of skill in the art. Elution methods can include two or more elution steps. Without wishing to be bound by theory, multiple elution steps can be used to first elute off contaminants (e.g., any other product other than Lactoferrin) and then elute off the desired product (e.g., Lactoferrin).

Elution methods can include two or more elution steps at different salt concentrations. Without wishing to be bound by theory, one or more initial elution steps (e.g., elution steps prior to the elution step containing the desired purified lactoferrin) can be performed to remove undesired proteins and other contaminants.

Elution methods can include a first elution step between 0.2-0.7M of a chemically pure NaCl solution. In general, a first elution step between 0.2-0.7M of a chemically pure NaCl solution is performed to remove undesired proteins and other contaminants. Without wishing to be bound by theory, a first elution can be monitored for completion by UV-Vis spectrometry and/or colorimetric assays monitoring the presence of contaminants such as lactoperoxidase and other enzymes native to the raw milk feed material. For example, a first elution step between 0.2-0.7M of a chemically pure NaCl solution can be performed until a lack of protein eluting off a resin is detected by UV-Vis spectrometry.

Elution methods can include a first elution step of 0.2M chemically pure NaCl solution. Elution methods can include a first elution step of 0.25M chemically pure NaCl solution. Elution methods can include a first elution step of 0.30M chemically pure NaCl solution. Elution methods can include a first elution step of 0.35M chemically pure NaCl solution. Elution methods can include a first elution step of 0.40M chemically pure NaCl solution. Elution methods can include a first elution step of 0.45M chemically pure NaCl solution. Elution methods can include a first elution step of 0.50M chemically pure NaCl solution. Elution methods can include a first elution step of 0.55M chemically pure NaCl solution. Elution methods can include a first elution step of 0.6M chemically pure NaCl solution. Elution methods can include a first elution step of 0.65M chemically pure NaCl solution. Elution methods can include a first elution step of 0.7M chemically pure NaCl solution. Elution methods can include a first elution step of a chemically pure NaCl solution below 1M.

Elution methods can include a second elution step of 1M chemically pure NaCl solution. In general, an elution using 1M chemically pure NaCl solution will elute lactoferrin from the resin. Elution methods can include a second elution step of about a 1M chemically pure NaCl solution.

Elution methods can include a first elution step between 0.2-0.7M of a chemically pure NaCl solution and a second elution step of 1M chemically pure NaCl solution. Elution methods can include a first elution step of 0.2M chemically pure NaCl solution and a second elution step of 1M chemically pure NaCl solution. Elution methods can include a first elution step of 0.25M chemically pure NaCl solution and a second elution step of 1M chemically pure NaCl solution. Elution methods can include a first elution step of 0.3M chemically pure NaCl solution and a second elution step of 1M chemically pure NaCl solution. Elution methods can include a first elution step of 0.35M chemically pure NaCl solution and a second elution step of 1M chemically pure NaCl solution. Elution methods can include a first elution step of 0.40M chemically pure NaCl solution and a second elution step of 1M chemically pure NaCl solution. Elution methods can include a first elution step of 0.45M chemically pure NaCl solution and a second elution step of 1M chemically pure NaCl solution. Elution methods can include a first elution step of 0.50M chemically pure NaCl solution and a second elution step of 1M chemically pure NaCl solution. Elution methods can include a first elution step of 0.55M chemically pure NaCl solution and a second elution step of 1M chemically pure NaCl solution. Elution methods can include a first elution step of less than 1M chemically pure NaCl solution and a second elution step of 1M chemically pure NaCl solution.

Elution methods can include a first elution step between 0.25-0.7M of a chemically pure NaCl solution and a second elution step of about a 1M chemically pure NaCl solution. Elution methods can include a first elution step of 0.20M chemically pure NaCl solution and a second elution step of about a 1M chemically pure NaCl solution. Elution methods can include a first elution step of 0.25M chemically pure NaCl solution and a second elution step of about a 1M chemically pure NaCl solution. Elution methods can include a first elution step of 0.30M chemically pure NaCl solution and a second elution step of about a 1M chemically pure NaCl solution. Elution methods can include a first elution step of 0.35M chemically pure NaCl solution and a second elution step of about a 1M chemically pure NaCl solution. Elution methods can include a first elution step of 0.40M chemically pure NaCl solution and a second elution step of about a 1M chemically pure NaCl solution. Elution methods can include a first elution step of 0.45M chemically pure NaCl solution and a second elution step of about a 1M chemically pure NaCl solution. Elution methods can include a first elution step of 0.50M chemically pure NaCl solution and a second elution step of about a 1M chemically pure NaCl solution. Elution methods can include a first elution step of 0.55M chemically pure NaCl solution and a second elution step of about a 1M chemically pure NaCl solution. Elution methods can include a first elution step of 0.60M chemically pure NaCl solution and a second elution step of about a 1M chemically pure NaCl solution. Elution methods can include a first elution step of 0.65M chemically pure NaCl solution and a second elution step of about a 1M chemically pure NaCl solution. Elution methods can include a first elution step of 0.70M chemically pure NaCl solution and a second elution step of about a 1M chemically pure NaCl solution.

Elution methods can include two or more elution steps at different pH levels. elution gradient. Elution methods can include two or more elution steps at different salt concentrations, different pH levels, and combinations thereof. Elution methods can include an elution gradient. Elution methods can include an salt elution gradient. Elution methods can include a pH elution gradient. Elution methods can include both a salt and a pH elution gradient.

For each of the above ion exchange chromatography steps (e.g., equilibration regeneration, loading, and/or elution) one of skill in the art will recognize the appropriate fluid velocities, e.g., as dependent on the choice of resin, apparatus, raw milk feed material, etc.

Purification methods can include filtration. Methods of filtration are known to those of skill in the art. Purification methods can include microfiltration (typically referring to filtration using a membrane pore size of 0.1 to 10 μm). Microfiltration can include a membrane pore size of 1-10 μm. Microfiltration can include a membrane pore size of 10 μm. Microfiltration can include a membrane pore size of 1-10 μm. Microfiltration can include a membrane pore size of 0.1-1 μm. Microfiltration can include a membrane pore size of 0.1 μm. Microfiltration can include a membrane pore size of 1 μm. Microfiltration can include a membrane pore size of 0.1, 0.2, 0.3, 0.3, 0.5, 0.6, 0.7, 0.8, 0.9, and/or 1 μm. Microfiltration can include a ceramic filter.

Purification methods can include ultrafiltration (typically referring to filtration using a membrane pore size of 0.01 to 0.1 μm). Ultrafiltration systems can also be referred to by molecular weight cutoff sizes designed for sized-based separation between the permeate and retentate. Ultrafiltration systems can include 5-30 kDa systems. Ultrafiltration systems can include a 5 kDa system. Ultrafiltration systems can include a 10 kDa system. Ultrafiltration systems can include a 15 kDa system. Ultrafiltration systems can include a 20 kDa system. Ultrafiltration systems can include a 25 kDa system. Ultrafiltration systems can include a 30 kDa system.

Purification methods can include both microfiltration and ultrafiltration, including in any order and/or separated be one or more addition purification processes. Purification methods can include multiple microfiltration and/or ultrafiltration steps, including in any order and/or separated be one or more addition purification processes. As an illustrative non-limiting example, a first pre-filtering microfiltration step (e.g., with a 10 μm filter) can be used prior to ion exchange chromatography, a second microfiltration step (e.g., with a 0.1-1.4 μm filter) can be used subsequent to ion exchange chromatography, followed by an ultrafiltration step (e.g., with a 5-30 kDa).

Purification methods can include a combination of chromatography and filtration, including in any order and/or separated be one or more addition purification processes. Purification methods can include a combination of multiple chromatography and/or filtration steps, including in any order and/or separated be one or more addition purification processes. Purification methods can include a combination of microfiltration, ultrafiltration, and ion exchange chromatography, including in any order and/or separated be one or more addition purification processes. In an illustrative non-limiting example, purification methods can include ion exchange chromatography (including elution), followed by microfiltration and then by ultrafiltration. In another illustrative non-limiting example, purification methods can include microfiltration, followed by ion exchange chromatography (including elution), next followed by additional microfiltration, and then by ultrafiltration.

Purification methods can include separation of a raw milk product into milk product derivatives, such as separating a natural milk source into skim milk and cream. For example, a raw milk product can be separated into milk product derivatives prior to chromatography and/or filtration. Methods of separating a raw milk product into milk product derivatives are known to those of skill in the art including, but not limited to, cold-bowl separation.

Following purification of Lactoferrin, the purified product can be dried. In general, drying methods provided do not include treatments that disrupt a native conformational state (e.g., denature Lactoferrin) and/or reduce Lactoferrin bioactivity with reference to conformational states or bioactivity, respectively, found in the natural milk source used for Lactoferrin purification. Methods of drying Lactoferrin are known to those of skill in the art including, but not limited to, freeze-drying/lyophilization, fluid-bed drying, and/or low-temperature spray-drying.

### Purity Assessment

Natural milk sources, including bovine milk, typically contain several protein components in addition to Lactoferrin including, but not limited to lactoperoxidase, lysozyme, caseins, immunoglobulins, lactalbumin, and lactoglobulin. Natural milk sources can also contain other components, such as fat and endotoxin. In general, the purification methods provided herein reduce, minimize, or eliminate components other than Lactoferrin. Without wishing to be bound by theory, removal of one or more of the additional components can improve Lactoferrin bioactivity and/or improve safety.

Provided herein are Lactoferrin compositions having an increased percentage of Lactoferrin by mass relative to the ratio present in an unprocessed Lactoferrin-comprising milk product.

Lactoferrin percentage can be assessed by mass-spectrometry including, but not limited to, matrix assisted laser desorption ionization-time of flight (MALDI-TOF) mass-spectrometry and/or Linear Trap Quadropole Orbitrap Velos mass-spectrometry. In general, assessment by mass-spectrometry comprises quantifying an area under a peak corresponding to Lactoferrin and areas under a peak that do not correspond to Lactoferrin. In some instances, Lactoferrin can be associated with multiple peaks, such as ionization peaks corresponding to Lactoferrin. Peaks corresponding to Lactoferrin can include peaks corresponding to a full-length post-translationally modified (e.g., glycosylated) Lactoferrin. Peaks corresponding to full-length post-translationally modified Lactoferrin generally range from about 80,000-86,000 m/z. The exact peak for a full-length post-translationally modified can change, such as reflecting different glycosylation statuses. In some instances, to be inclusive, peaks having an m/z between 79000-90000 can be considered corresponding to Lactoferrin. Areas under a peak that do not correspond to Lactoferrin include all other peaks, with the potential exception of an ionization peak associated with Lactoferrin around 41,500 m/z (e.g., having an m/z between 41000-42000). Areas under a peak that do not correspond to Lactoferrin can include peaks having an m/z between 18000-80000 (other than peaks having an m/z between 41000-42000). A particular comparison can also be made between Lactoferrin and peaks corresponding to Lactoperoxidase (e.g., peaks having an m/z between 77000 and 78000). Areas under a peak that do not correspond to Lactoferrin can include peaks having an m/z between 18000-45000 (other than peaks having an m/z between 41000-42000). Linear Trap Quadropole Orbitrap Velos mass-spectrometry can also quantify the percentage of Lactoferrin relative to other components in a sample. For example, Quadropole Orbitrap Velos mass-spectrometry can quantify the percentage of Lactoferrin through determining the percentage of Peptide Spectrum Matches (PSMs).

Lactoferrin percentage can be assessed by liquid chromatography, such as high-performance liquid chromatography (HPLC). Assessment by liquid chromatography can include quantifying an area under a peak corresponding to Lactoferrin and areas under a peak that do not correspond to Lactoferrin.

For assessment involving quantification of peaks, one or more of the areas under a peak that do not correspond to Lactoferrin present in an unprocessed Lactoferrin-comprising milk product can be below a limit of detection for the purified Lactoferrin composition. In some instances, each area under a peak that does not correspond to Lactoferrin can be below a limit of detection.

Lactoferrin percentage can be assessed by an enzyme-linked immunosorbent assay (ELISA). In some instances, an ELISA can distinguish the percentage of Lactoferrin in a native protein conformation, such as by using an antibody that specifically binds the native protein conformation of Lactoferrin.

A specific component typically present in unprocessed milk sources to be reduced, minimized, or eliminated when purifying Lactoferrin is Lactoperoxidase. In addition, Lactoperoxidase is frequently present in detectable amounts in purified Lactoferrin compositions produced by typical industrial methods.

Provided herein are Lactoferrin compositions where at least at least 70% of the composition is purified Lactoferrin. Compositions include those where at least 75%, at least 80%, at least 85%, at least 90%, or at least 95% of the composition is purified Lactoferrin. Compositions include those where at least 75% of the composition is purified Lactoferrin. Compositions include those where at least 80% of the composition is purified Lactoferrin. Compositions include those where at least 85% of the composition is purified Lactoferrin. Compositions include those where at least 90% of the composition is purified Lactoferrin. Compositions include those where at least 95% of the composition is purified Lactoferrin. Compositions include those where at least about 100% of the composition is purified Lactoferrin. Compositions include those where at least 91%, at least 92%, at least 93%, at least 94%, at least 95%, at least 96%, at least 97%, at least 98%, or at least 99% of the composition is purified Lactoferrin. Compositions include those where at least 91% of the composition is purified Lactoferrin. Compositions include those where at least 92% of the composition is purified Lactoferrin. Compositions include those where at least 93% of the composition is purified Lactoferrin. Compositions include those where at least 94% of the composition is purified Lactoferrin. Compositions include those where at least 95% of the composition is purified Lactoferrin. Compositions include those where at least 96% of the composition is purified Lactoferrin. Compositions include those where at least 97% of the composition is purified Lactoferrin. Compositions include those where at least 98% of the composition is purified Lactoferrin. Compositions include those where at least 99% of the composition is purified Lactoferrin.

Provided herein are Lactoferrin compositions having an increased ratio of Lactoferrin: Lactoperoxidase relative to the ratio in an unprocessed Lactoferrin-comprising milk product. Ratios of Lactoferrin: Lactoperoxidase can be assessed according to methods known to those skilled in the art, such as the purity assessment methods described herein (e.g., mass-spectrometry, HPLC, and/or ELISA). For example, assessment can include quantifying an area or areas under a peak corresponding to Lactoferrin and an area under a peak corresponding to Lactoperoxidase.

Endotoxin present or potentially present in natural milk sources can be reduced, minimized, or eliminated. Without wishing to be bound by theory, removal of endotoxin can improve the safety of a purified Lactoferrin composition. Methods of assessing endotoxin levels are known to those skilled in the art.

Also provided for herein are methods of assessing purity of a purified Lactoferrin composition, such as using any of the purity assessment methods described herein.

### Pharmaceutical Composition

Provided herein are pharmaceutical compositions comprising any one of the purified Lactoferrin compositions described herein and one or more pharmaceutically acceptable excipients.

As used herein, a “pharmaceutical composition” is meant to encompass a composition suitable for administration to a subject, such as a mammal, especially a human. In general a “pharmaceutical composition” is sterile, and preferably free of contaminants that are capable of eliciting an undesirable response within the subject (e.g., the compound(s) in the pharmaceutical composition is pharmaceutical grade). Pharmaceutical compositions can be designed for administration to subjects or patients in need thereof via a number of different routes of administration including, but not limited to, topical administration routes such as skin (e.g., wound), mucosal, respiratory, oral (including gastrointestinal), and nasal formulations.

A “pharmaceutically acceptable excipient,” “pharmaceutically acceptable diluent,” “pharmaceutically acceptable carrier,” and “pharmaceutically acceptable adjuvant” means an excipient, diluent, carrier, and adjuvant that are useful in preparing a pharmaceutical composition that are generally safe, non-toxic and neither biologically nor otherwise undesirable, and include an excipient, diluent, carrier, and adjuvant that are acceptable for veterinary use as well as human pharmaceutical use. “A pharmaceutically acceptable excipient, diluent, carrier and adjuvant” as used in the specification and claims includes both one and more than one such excipient, diluent, carrier, and adjuvant.

For pharmaceutical applications, Lactoferrin may be incorporated in an orally dissolvable formulation. This may include, but is not limited to, orally dissolvable tablets, lozenges, gum, oral thin film, or direct application of lactoferrin in an excipient powder. Pharmaceutical acceptable excipients include, but are not limited to, sodium steryl fumate, citric acid, xylitol (Xylisorb XTAB 240), sorbitol (Neosorb XTAB 200S or Neosorb P60 W), mannitol, maltitol (Sweetpearl P300 DC), and/or other sugar alcohols, including any combinations thereof. Without wishing to be bound by theory, orally dissolvable formulations can promote retention of lactoferrin in the mouth and nasopharynx for local targeting of the lactoferrin. Orally dissolvable formulation may be prepared using techniques known to the art, such as fluid bed drying or any other drying techniques coupled to pressing the product into a final dosage form. Oral formulations can include bio-adhesives. Applicable bio-adhesives are known to those skilled in the art, such as those described in Duan et al. (Applications of Bioadhesives: A Mini Review; Front Bioeng Biotechnol. 2021; 9: 716035.), herein incorporated by reference for all purposes. Oral formulations can include an astringent. Applicable astringents are known to those skilled in the art including, but not limited to, alum, acacia, sage, yarrow, witch hazel, bayberry, distilled vinegar, persimmon, green tea, black tea, citric acid, cranberry juice and/or extract, grapefruit juice and/or extract, or inorganic acids.

Oral formulations can have particular applicability for gastrointestinal applications. Oral formulations, including for gastrointestinal applications, can include microencapsulation of Lactoferrin. Oral formulations, including for gastrointestinal applications, can include microencapsulation of pharmaceutical compositions comprising any one of the purified Lactoferrin compositions described herein, including enteric coated formulations. Oral formulations, including for gastrointestinal applications, can include microencapsulation (e.g., enteric coated multi-ingredient formulations) of pharmaceutical compositions comprising any one of the purified Lactoferrin compositions in combination with one or more additional active agents. Microencapsulation formats are known to those skilled in the art, such as those described in Singh et al. (Microencapsulation: A promising technique for controlled drug delivery; Res Pharm Sci. 2010 Jul.-Dec; 5(2): 65-77.), herein incorporated by reference for all purposes.

For pharmaceutical applications directed to wound healing, Lactoferrin may be incorporated in formulations that promote contact of Lactoferrin with a wound. Wounds can be any damage to a subject's tissue. Damage includes, but is not limited to, burns (e.g., fire/thermal, chemical, electrical, radiation [such as ultraviolet or radioactive], friction etc.), cuts (e.g., punctures, lacerations, abrasions, incisions [such as surgical incisions], amputations, etc.), infections, and ulcerations. Wounds can include open wounds. Wounds can include ulcers, such as a venous or arterial ulcer. Multiple forms of damage can exist concurrently. For example, as illustrative non-limiting examples, damage can include both a burn together with an infection or a cut together with an infection. Tissues include, but are not limited to, skin and lung tissue. Damage to multiple tissues can exist concurrently. For example, as an illustrative non-limiting example, damage can include damage to both skin and lung tissue. Formulations that promote contact of lactoferrin with a wound can include bio-adhesives. Applicable bio-adhesives are known to those skilled in the art, such as those described herein. Delivery formats for wound-healing formulations include, but are not limited to, gelfoams, hydrogels, infused bandages, powders, sprays, and inhalation formats (e.g., nebulizer, inhaler, and misting formats).

For pharmaceutical applications directed to promoting anti-microbial uses, Lactoferrin may be incorporated in formulations that promote anti-microbial activities of Lactoferrin. Anti-microbial uses can include treatment of an active infection. Anti-microbial uses can include prophylactic prevention and/or treatment of an infection. For example, prophylactic prevention and/or treatment of an infection can include application to a wound before infection results.

Formulations and/or delivery formats can promote multiple uses and/or properties of Lactoferrin. As a non-limiting illustrative example, a formulation can promote both wound healing and anti-microbial properties of Lactoferrin, such as a formulation or delivery format that can promote both cell growth and prevent microbial biofilm formation.

Lactoferrin may also be incorporated into formulations typically regarded as foods for purpose of medication of these products, referred to as “medical foods.” These applications include incorporation in gum or other chewable products for slow release of Lactoferrin. Medical food formulations, such as gum, may include excipients such as xylitol, powdered sugar, gum base, sorbitol, mannitol, sucralose, and/or corn syrup, including in any combination. For gum, Lactoferrin may be incorporated either throughout the gum or as a coating on the exterior of the gum. Medical food formulations may be prepared using techniques known to the art such as fluid bed drying or any other drying technique and a final preparation with the gum base to form a final dosage form.

Formulations can include sodium steryl fumate, carbomer, sodium hydroxide, glycerin, sodium benzoate, benzoic acid, citric acid, and/or combinations thereof. Formulations can include xylitol, sorbitol, mannitol, maltitol, sugar alcohols, sucralose, and/or combinations thereof. Formulations can include Isomalt, microcrystalline cellulose, Sodium Carboxymethylcellulose, and/or combinations thereof. Formulations can include powdered sugar, gum base, corn syrup, gum Arabic, and/or combinations thereof. Formulations can include HiG PWD-04, Encapsulated powdered flavoring, Anhydrous or Natural Caffeine, Encapsulated Sucralose or Sucralose, Encapsulated Acesulfame-K and Aspartame, Aspartame, Acesulfame-K, Triacetin, Silicon dioxide, and/or combinations thereof.

Formulations can include a coagulant, including, but not limited to, thrombin, amylopectin, kaolin, and/or combinations thereof.

Pharmaceutical compositions, including orally dissolvable formulations and medical foods, may have optimized pH to maximize the stability and activity of the protein. The dosage format, including the use of select excipients, may contribute to enhancing the bioavailability, stability, and/or bioactivity of the protein.

Also provided herein are compositions that can take the format described herein of any of the pharmaceutical compositions described herein but are not pharmaceutical in nature, such as cosmetics (e.g., skin or oral cosmetic products).

### Methods of Treatment and Risk Prevention/Reduction

Provided herein are methods of treating a disease or condition through administering a therapeutically effective amount of any the pharmaceutical compositions described herein, such as any one of the pharmaceutical compositions formulated for delivery to an oral cavity, a nasal cavity, or other tissues (e.g., skin or lung tissue). Diseases or conditions include, but are not limited to, pathogenic diseases, gastrointestinal (GI) infections, wound infections, allergies, and inflammatory conditions.

Also provided herein are methods of modulating an immune response (e.g., promoting anti-inflammatory activity) through administering a therapeutically effective amount of any one of the purified Lactoferrin compositions described herein, including administering any of the pharmaceutical compositions described herein. The term “modulate” encompasses maintenance of a biological activity, inhibition (partial or complete) of a biological activity, and stimulation/activation (partial or complete) of a biological activity. The term also encompasses decreasing or increasing (e.g., enhancing) a biological activity. For example, administering a therapeutically effective amount of purified Lactoferrin can reduce inflammation through promoting anti-inflammatory activity, e.g., in the context of an inflammatory disease.

As used herein, the terms “treatment,” “treating,” and the like, refer to obtaining a desired pharmacologic and/or physiologic effect, such as prevention, risk reduction, amelioration and/or resolution of an infection, such as a viral, fungal (e.g., yeast), or bacterial infection. Treatments may be a prophylactic treatment in terms of completely or partially preventing a disease or symptom thereof and/or may be therapeutic in terms of a partial or complete cure for a disease and/or adverse effect attributable to the disease. Treatment covers any treatment of a disease in a mammal, particularly in a human, and includes: (a) prophylactically treating (completely or partially preventing) the disease or a symptom of a disease from occurring in a subject which may be predisposed to the disease but has not yet been diagnosed as having it (e.g., as in a subject at risk for an infection); (b) inhibiting the disease (e.g., eliminating an infection and/or reducing it below a detectable limit); and (c) relieving the disease (e.g., reducing microbial burden associated with an infection).

Also provided herein are methods for reducing the risk of a pathogenic disease, the method comprising administering any of the pharmaceutical compositions or compositions described herein to a subject. For example, reducing risk for a pathogenic disease can include, but is not limited to, administration the pharmaceutical compositions or compositions described herein to a wound at risk for a pathogenic disease, e.g., an infection by a pathogen. A subject at risk for a pathogenic disease can have been exposed to a pathogen. A subject at risk for a pathogenic disease can have been diagnosed with an infection by a pathogen. Reducing risk can be include administration of pharmaceutical compositions or compositions described herein that reduce the risk of a pathogenic disease relative to a subject who has not been administered the pharmaceutical compositions or compositions.

Also provided herein are methods for treating a subject identified as being at risk for a pathogenic disease, the method comprising administering any of the pharmaceutical compositions or compositions described herein to the subject thereby reducing the risk of the pathogenic disease.

A “therapeutically effective amount” or “efficacious amount” means the amount of a compound that, when administered to a mammal or other subject for treating a disease, condition, or disorder, is sufficient to effect such treatment for the disease, condition, or disorder. The “therapeutically effective amount” will vary depending on the compound, the disease and its severity and the age, weight, etc., of the subject to be treated.

The subject compounds can be administered to a subject alone or in combination with an additional active agent. The terms “agent,” “compound,” and “drug” are used interchangeably herein. The method can further include co-administering concomitantly or in sequence a second agent, e.g., a small molecule, an antibody, an antibody fragment, an antibody-drug conjugate, an aptamer, a protein, an antibiotic, an anti-viral agent, an anti-microbial agent, an anti-bacterial agent, an anti-fungal agent, and/or a vaccine. In some embodiments, the method further includes performing radiation therapy on the subject.

The terms “co-administration” and “in combination with” include the administration of two or more therapeutic agents either simultaneously, concurrently or sequentially within no specific time limits. In one embodiment, the agents are present in the cell or in the subject's body at the same time or exert their biological or therapeutic effect at the same time. In one embodiment, the therapeutic agents are in the same composition or unit dosage form. In other embodiments, the therapeutic agents are in separate compositions or unit dosage forms. The term “unit dosage form,” as used herein, refers to physically discrete units suitable as unitary dosages for human and animal subjects, each unit containing a predetermined quantity of a compound (e.g., an aminopyrimidine compound, as described herein) calculated in an amount sufficient to produce the desired effect in association with a pharmaceutically acceptable diluent, carrier or vehicle. The specifications for unit dosage forms depend on the particular compound employed and the effect to be achieved, and the pharmacodynamics associated with each compound in the host. In certain instances, the combination provides an enhanced effect relative to either component alone; in some cases, the combination provides a supra-additive or synergistic effect relative to the combined or additive effects of the components. For multiple dosages, the two agents may directly alternate, or two or more doses of one agent may be alternated with a single dose of the other agent, for example.

Also provided herein is a method of treating a pathogenic disease. The method can include administering a pharmaceutical compositions to a subject, such as any one of the pharmaceutical compositions described herein (e.g., any one of the pharmaceutical compositions formulated for delivery to an oral cavity or nasal cavity). Administration can be prophylactic. A subject can be at risk for exposure to a pathogen. For example, risk for exposure to a pathogen can include, but is not limited to, administration to a wound at risk for an infection by a pathogen. A subject can have been exposed to a pathogen. A subject can have been diagnosed with an infection by a pathogen.

Pathogens include, but are not limited to, orally and/or nasally transmitted microbes. Pathogens include, but are not limited to, airborne microbes. Pathogens include, but are not limited to, a virus, such as a coronavirus (e.g., SARS-COV-2.) Pathogens include, but are not limited to, a bacterium and/or a fungus (e.g., a yeast).

Also provided herein are methods of promoting wound healing (e.g., through promoting anti-inflammatory activity, promoting cell growth, and/or preventing infection) through administering a therapeutically effective amount of any one of the purified Lactoferrin compositions described herein, including administering any of the pharmaceutical compositions described herein.

### Methods of Manufacturing

Provided herein are methods of any one of the purified Lactoferrin compositions described herein, including any of the pharmaceutical compositions described herein. Methods include any one of the purification steps described in the section “Milk Product Purification,” including combinations of the steps described therein. Illustrative non-limiting examples are described in the Examples section herein.

### 7. EXAMPLES

Below are examples of specific embodiments for carrying out the present invention. The examples are offered for illustrative purposes only, and are not intended to limit the scope of the present invention in any way. Efforts have been made to ensure accuracy with respect to numbers used (e.g., amounts, temperatures, etc.), but some experimental error and deviation should, of course, be allowed for.

The practice of the present invention will employ, unless otherwise indicated, conventional methods of protein chemistry, biochemistry, recombinant DNA techniques and pharmacology, within the skill of the art. Such techniques are explained fully in the literature. See, e.g., T. E. Creighton, Proteins: Structures and Molecular Properties (W. H. Freeman and Company, 1993); A. L. Lehninger, Biochemistry (Worth Publishers, Inc., current addition); Sambrook, et al., Molecular Cloning: A Laboratory Manual (2nd Edition, 1989); Methods In Enzymology (S. Colowick and N. Kaplan eds., Academic Press, Inc.); Remington's Pharmaceutical Sciences, 18th Edition (Easton, Pennsylvania: Mack Publishing Company, 1990); Carey and Sundberg Advanced Organic Chemistry 3rd Ed. (Plenum Press) Vols A and B (1992).

### Example 1. Purification of Lactoferrin

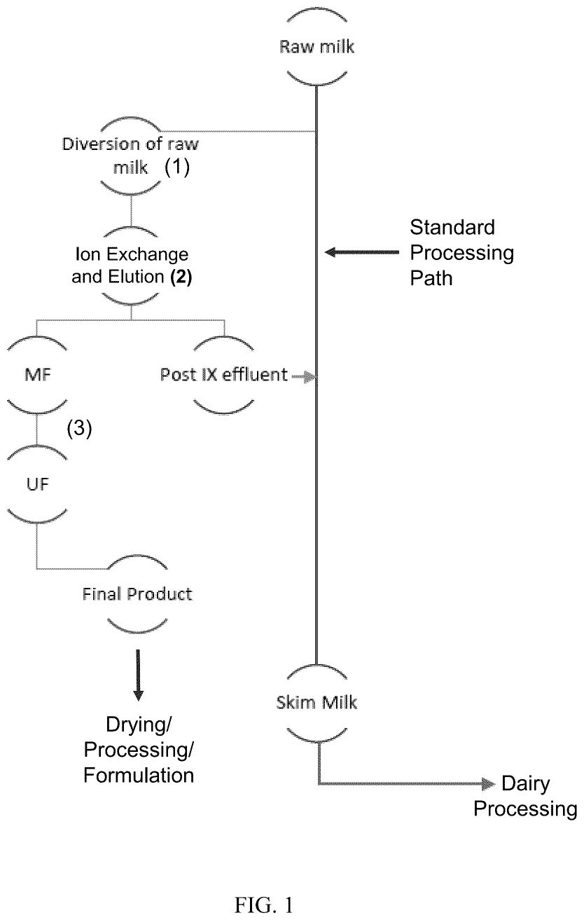

Lactoferrin was purified as illustrated in FIG. 1. Briefly, raw milk (untreated milk, e.g., not chemically, enzymatically, acid, or heat treated prior to purification of the Lactoferrin) was (1) diverted prior to entry into an industry standard milk processing workflow; (2) flowed through an ion exchange resin/column with effluent typically returned to the standard milk processing workflow and Lactoferrin-containing eluates collected; (3) collected eluates filtered; and (4) purified Lactoferrin processed. Notably, all other known Lactoferrin purification processes begin with a source of milk obtained after the industry standard milk processing workflow that typically includes heat pasteurization. In addition, a natural unprocessed milk product (e.g., raw milk) was used as a source material so that Lactoferrin included relevant post-translation modifications potentially involved in bioactivity, in contrast to recombinantly-produced Lactoferrin.

A chromatography column was used to isolate and purify bovine lactoferrin to retain its native post-translational modifications, glycosylation and bound iron. The column was loaded with the chosen cation exchange resin, either polymethacrylate or agarose matrix, and equilibrated and regenerated by rinsing with (1) reverse osmosis water; (2) chemically pure 1M NaCl; and (3) rinsed again with reverse osmosis water.

Raw, untreated, unprocessed bovine whole milk (less than 24 hrs. since milking) was obtained directly from a dairy transport or silo vessel before being separated, skimmed, heated and/or pasteurized. Lactoferrin was purified from both raw colostrum (RC) and raw whole milk (RM).

The milk was filtered for large particulates with a 10 μm filter and was warmed to above 37° C. and kept at a temperature below 63° C. (generally considered the starting temperature for pasteurization) and loaded into the chromatography column. The volume loaded was based on a pre-determined binding capacity and an estimation of the native Lactoferrin content of the raw milk feed material. During the loading process, all flow-through was returned to the pasteurizer balance tank to return the feed to the factory to the point prior to separation and pasteurization.

Once loading was completed, the resin was rinsed with reverse osmosis water to remove any remaining milk compounds which have not bound to the resin. A 1^{st} elution included washing of the resin with a chemically pure NaCl solution (0.2-0.7M) and was monitored and assessed for completion (i.e., a lack of protein eluting off the resin) by UV-Vis spectrometry and colorimetric assays monitoring the presence of contaminants such as lactoperoxidase and other enzymes native to the raw milk feed material. In particular, the 1^{st} NaCl was performed until lactoperoxidase, generally the major contaminant, was no longer present in the eluted fractions as assessed by peroxidase colorimetric assay using a colorimetric peroxidase substrate. The fraction of the 1^{st} elution was retained in an isolated vessel. The resin was then rinsed with reverse osmosis water. The 2^{nd} elution was performed with 1M NaCl to remove the isolated Lactoferrin. The fraction of the 2^{nd} elution was retained in an isolated vessel and then (1) filtered through a ceramic microfiltration filter (0.1-1.4 μm); and (2) concentrated using an ultrafiltration system (5-30 kDa).

The desalinated eluent was then freeze dried but may also proceed directly into a fluid bed dryer to be dried onto a pharmaceutical grade excipient. The purified lactoferrin produced according to the methods described herein are referred to as: Hyacinth lactoferrin (“Hyacinth”); Lactoferrin (solely without the accompanying words “Lab-grade” or “Commercially available supplement grade”; ODT-SC210; and API-E2. The various names may refer to purified lactoferrin produced according to variations of the methods described herein.

### Example 2. Assessment of Lactoferrin Purity

Purified Lactoferrin produced according to Example 1 was assessed for purity.

Typically, SDS-PAGE gels are used to assess purity for reference grade protein-derived products, including reference grade Lactoferrin. However, SDS-PAGE gels are notorious for producing exaggerated purity results depending on experimental conditions (Kurien and Scofield Methods Mol Biol. 2012; 869: 633-640). Accordingly, the more sensitive methods of mass-spectrometry, HPLC, and ELISA were used to assess purity.

Lactoferrin purity was assessed by MALDI-TOF mass-spectrometry. Dried samples were dissolved in water at a concentration of 10 mg/mL. Dissolved samples were mixed with an equal volume of saturated sinapinic acid in 50% acetonitrile containing 0.1% trifluoracetic acid. The sample/matrix mixture (2 μL) was placed on an MIP 384 ground steel MALDI plate. MALDI mass spectra were acquired at m/z 2001-20162 Da (2-20 kDa), 10039-40026 Da (10-40 kDa), and 19780-100000 Da (20 kDa-100 kDa) in positive ion mode. The instrument was calibrated in these mass ranges using Protein Calibration Standard II (Bruker). MS spectra were analyzed using FlexAnalysis 3.4 (Bruker Daltonics, Billerica, MA).

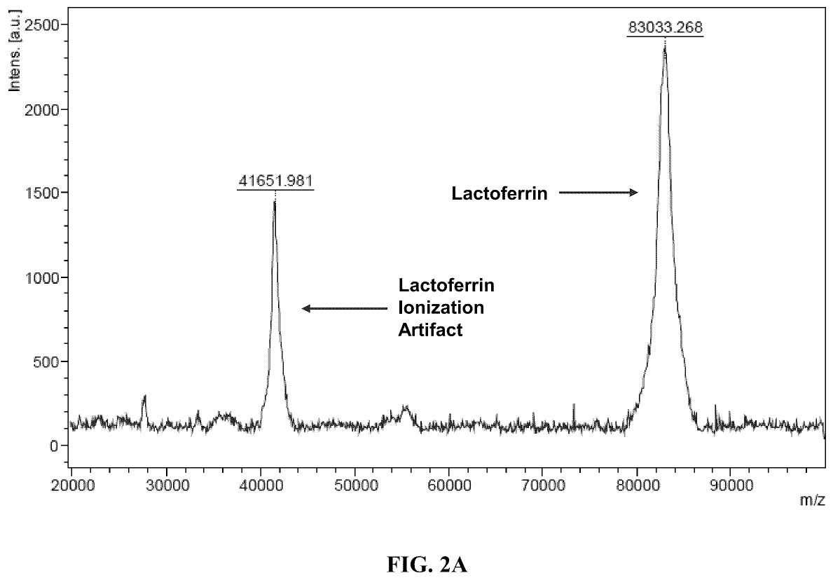

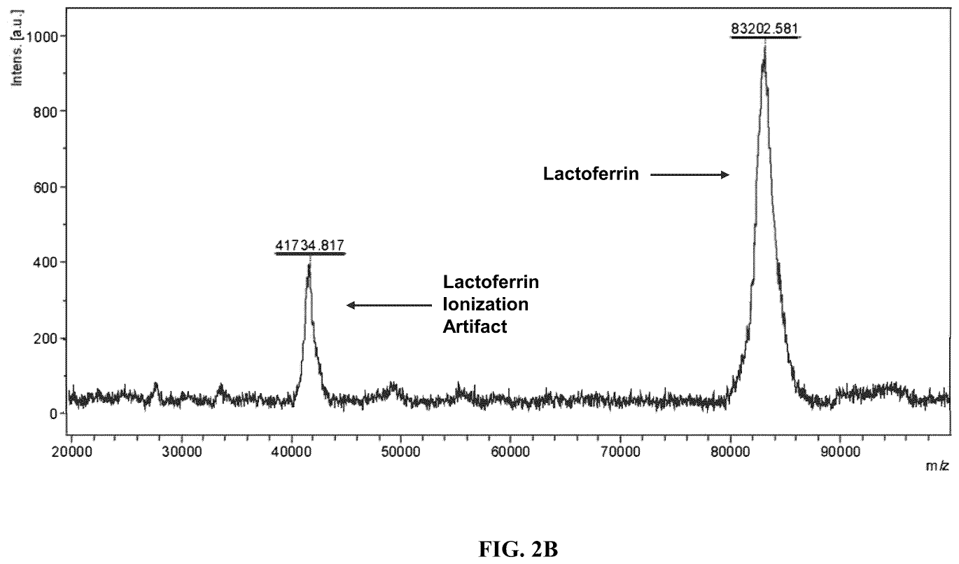

Purity results as assessed by MALDI-TOF (˜18 kDa-100 kDa) for Lactoferrin purified according to Example 1 from two different natural raw milk sources (colostrum and whole milk) are shown in FIG. 2A and FIG. 2B, and quantified in Table 1A and Table 1B, respectively. The dominant peaks above ˜80,000 m/z correspond to glycosylated Lactoferrin, while the peaks at ˜41,500 m/z correspond to ionization peaks of Lactoferrin. Notably, the typical contamination peak associated with Lactoperoxidase (˜78,000 m/z) was below the limit of detection. Quantification of the mass-spectrometry profiles revealed that greater than 75% of the relative area under the curve (AUC) determined for the quantitated peaks in the range of 18 kDa-100 kDa corresponded to the desired glycosylated Lactoferrin peak between 80-85 kDa, and close to 100% corresponded to Lactoferrin when including the ionization peak at ˜41,500 m/z (peaks selected for quantification, in other words considered true peaks above background noise, were determined by FlexAnalysis 3.4). Accordingly, the results demonstrate the purification process described herein produced highly pure Lactoferrin from various raw natural milk products.

TABLE 1A
Quantification of MALDI-TOF (Raw Colostrum)

| m/z | S/N | Intens. | Area | Relative Peak Area (%) |
|---|---|---|---|---|
| 41651.98 | 11 | 1344 | 1.53 × 10⁶ | 22.95 |
| 83033.27 | 20 | 2266 | 5.14 × 10⁶ | 77.05 |

TABLE 1B
Quantification of MALDI-TOF (Raw Whole Milk)

| m/z | S/N | Intens. | Area | Relative Peak Area (%) |
|---|---|---|---|---|
| 41734.817 | 9 | 374 | 4.17 × 10⁵ | 16.95 |
| 83202.581 | 22 | 882 | 2.04 × 10⁶ | 83.05 |

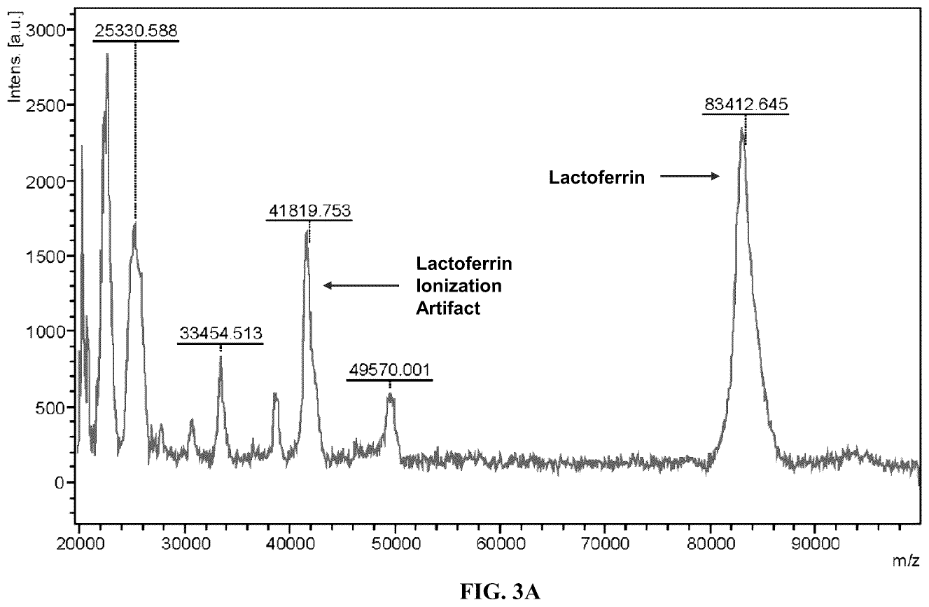

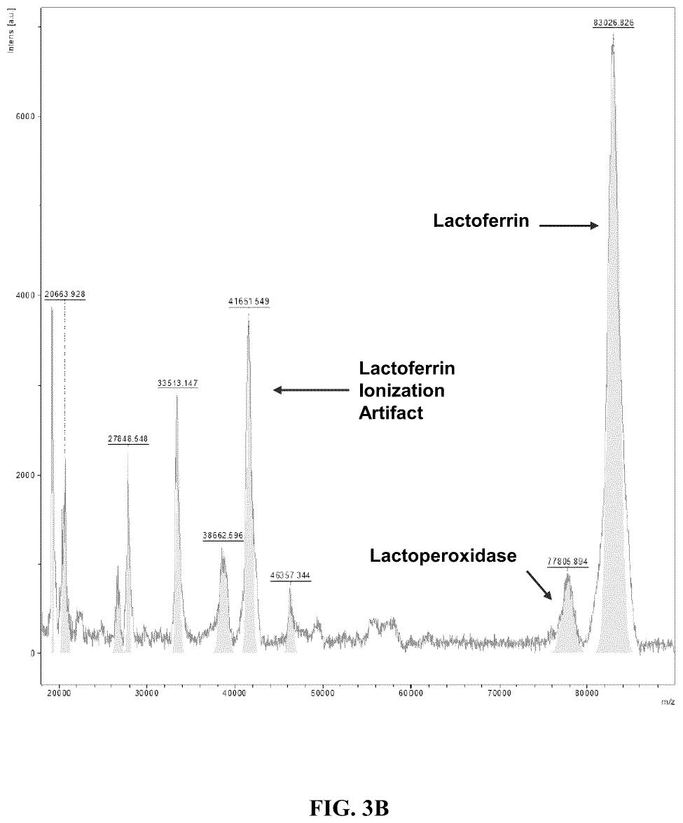

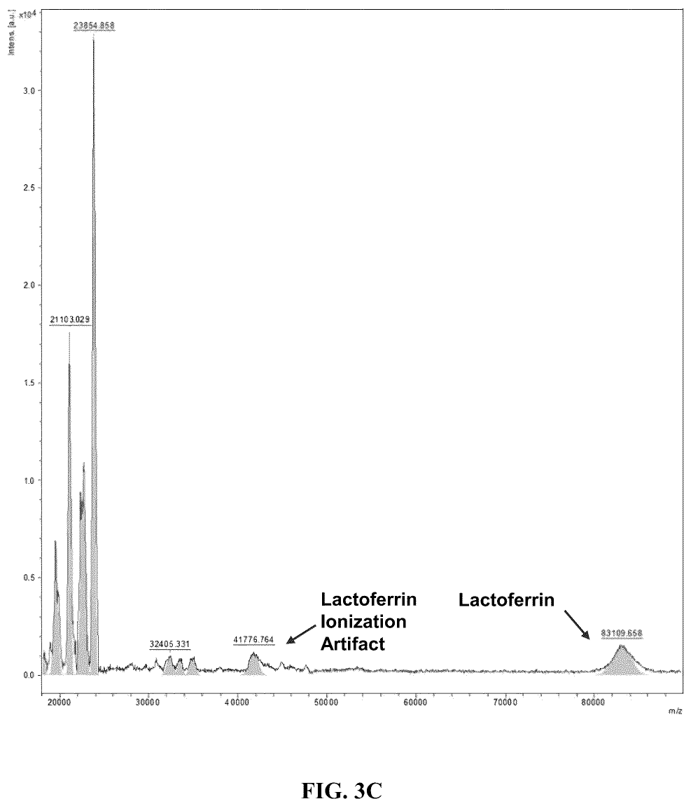

Purity was also assessed by MALDI-TOF (˜18 kDa-100 kDa) for an over the counter (OTC) Lactoferrin supplement [Jarrow Formulas] (FIG. 3A) and for Lactoferrin sources advertised as laboratory reagent grade products [Sigma Bovine Colostrum Lactoferrin] (FIG. 3B and FIG. 3C), as above, and quantified in Table 2A, Table 2B, and Table 2C, respectively. In contrast to the Lactoferrin produced according to Example 1, mass-spectrometry profiles reveal contaminating peaks for the OTC supplement and both laboratory reagent grade Lactoferrin sources, in particular Lactoperoxidase (see FIG. 3B peak at 77806 m/z). Quantification of the mass-spectrometry profiles revealed that only 25.3%, 52.3%, and 11.9%, respectively, of the relative AUCs determined for the quantitated peaks in the range of 18 kDa-100 kDa corresponded to the desired glycosylated Lactoferrin peak between 80-85 kDa. Even accounting for the Lactoferrin ionization peak at ˜41,500 m/z, only combined relative AUCs of 33.5%, 66.4%, and 15.9%, respectively, corresponded to Lactoferrin. Accordingly, the results demonstrate the purification process described herein produced Lactoferrin at a higher purity than available OTC supplements and laboratory reagent grade Lactoferrin sources.

TABLE 2A
Quantification of MALDI-TOF for OTC Supplement (FIG. 3A)

| m/z | S/N | Intens. | Area | Relative Peak Area (%) |
|---|---|---|---|---|
| 20345.22 | 12 | 2229 | 452079 | 2.2 |
| 20708.48 | 6 | 1107 | 203598 | 1.0 |
| 22599.98 | 16 | 2835 | 2.30E+06 | 11.4 |
| 22607.87 | 16 | 2835 | 2.30E+06 | 11.4 |
| 22610.23 | 16 | 2835 | 2.30E+06 | 11.4 |
| 22612.58 | 16 | 2835 | 2.30E+06 | 11.4 |
| 25330.59 | 10 | 1717 | 2.10E+06 | 10.5 |
| 33454.51 | 5 | 830 | 462461 | 2.3 |
| 38691.72 | 4 | 579 | 372891 | 1.9 |
| 41819.75 | 10 | 1559 | 1.65E+06 | 8.2 |
| 49570 | 4 | 593 | 583820 | 2.9 |
| 83412.65 | 17 | 2204 | 5.08E+06 | 25.3 |

TABLE 2B
Quantification of MALDI-TOF for
Commercial Reagent #1 (FIG. 3B)

| m/z | S/N | Area | Relative Peak Area (%) |
|---|---|---|---|
| 19287 | 16 | 1.05E+06 | 4.6 |
| 20664 | 9 | 948774 | 4.1 |
| 26702 | 4 | 381374 | 1.6 |
| 27849 | 9 | 878698 | 3.8 |
| 33513 | 12 | 1.64E+06 | 7.1 |
| 38663 | 5 | 1.13E+06 | 4.9 |
| 41652 | 16 | 3.27E+06 | 14.1 |
| 46357 | 3 | 453094 | 2.0 |
| 77806 | 5 | 1.27E+06 | 5.5 |
| 83027 | 37 | 1.21E+07 | 52.3 |

TABLE 2C
Quantification of MALDI-TOF for
Commercial Reagent #2 (FIG. 3C)

| m/z | S/N | Area | Relative Peak Area (%) |
|---|---|---|---|
| 18235 | 4 | 270406 | 0.7 |
| 19561 | 21 | 3.52E+06 | 9.6 |
| 21103 | 53 | 6.43E+06 | 17.6 |
| 22602 | 31 | 7.13E+06 | 19.5 |
| 23855 | 99 | 1.16E+07 | 31.6 |
| 32405 | 3 | 747098 | 2.0 |
| 33527 | 3 | 471200 | 1.3 |
| 34932 | 3 | 641140 | 1.8 |
| 41777 | 5 | 1.45E+06 | 4.0 |
| 83110 | 10 | 4.37E+06 | 11.9 |

Lactoferrin purity was also assessed by Linear Trap Quadropole Orbitrap Velos mass-spectrometry. As shown in FIG. 4 and quantified in Table 3A, the Orbitrap Velos mass-spectrometry profile for Lactoferrin produced according to Example 1 revealed that greater than 80% of the identified Peptide Spectrum Matches (PSMs) corresponded to the desired Lactoferrin. In contrast, as shown in Table 3B, the Orbitrap Velos mass-spectrometry profile for the OTC supplement revealed that only ˜62% of the identified PSMs corresponded to Lactoferrin. Notably, ˜11% of the identified PSMs corresponded to the major contaminant lactoperoxidase. Accordingly, the results demonstrate the purification process described herein produced highly pure Lactoferrin and at a higher purity than other available Lactoferrin sources.

TABLE 3A
Quantification of Velos/Orbitrap (FIG. 4)

| Protein | % Coverage | # PSM | % Total |
|---|---|---|---|
| Lactotransferrin OS = Bos taurus OX = 9913 | 71 | 517 | 82.19 |
| GN = LTF PE = 1 SV = 2 | | | |
| Complement factor B OS = Bos taurus OX = 9913 | 33 | 25 | 3.97 |
| GN = CFB PE = 1 SV = 2 | | | |
| Chitinase-3-like protein 1 OS = Bos taurus OX = 9913 | 46 | 24 | 3.82 |
| GN = CHI3L1 PE = 1 SV = 3 | | | |
| Beta-1,4-galactosyltransferase 1 OS = Bos taurus | 33 | 19 | 3.02 |
| OX = 9913 GN = B4GALT1 PE = 1 SV = 3 | | | |
| Serotransferrin OS = Bos taurus OX = 9913 GN = TF | 5 | 18 | 2.86 |
| PE = 2 SV = 1 | | | |
| Polymeric immunoglobulin receptor OS = Bos taurus | 22 | 14 | 2.23 |
| OX = 9913 GN = PIGR PE = 2 SV = 1 | | | |
| Beta-2-microglobulin OS = Bos taurus OX = 9913 | 49 | 12 | 1.91 |
| GN = B2M PE = 1 SV = 2 | | | |

TABLE 3B
Quantification of Velos/Orbitrap for OTC Supplement

| Protein | % Coverage | # PSM | % Total |
|---|---|---|---|
| Lactotransferrin OS = Bos taurus OX = 9913 | 78 | 693 | 61.93 |
| GN = LTF PE = 1 SV = 2 | | | |
| Lactoperoxidase OS = Bos taurus OX = 9913 | 50 | 121 | 10.81 |
| GN = LPO PE = 1 SV = 1 | | | |
| Serotransferrin OS = Bos taurus OX = 9913 GN = TF | 52 | 69 | 6.17 |
| PE = 2 SV = 1 | | | |
| Folate receptor alpha OS = Bos taurus OX = 9913 | 49 | 38 | 3.40 |
| GN = FOLR1 PE = 1 SV = 3 | | | |
| Beta-lactoglobulin OS = Bos taurus OX = 9913 | 47 | 25 | 2.23 |
| GN = LGB PE = 1 SV = 3 | | | |
| Angiogenin-1 OS = Bos taurus OX = 9913 | 37 | 21 | 1.88 |
| GN = ANG1 PE = 1 SV = 4 | | | |
| Cystatin-C OS = Bos taurus OX = 9913 GN = CST3 | 45 | 19 | 1.70 |
| PE = 1 SV = 2 | | | |
| Albumin OS = Bos taurus OX = 9913 GN = ALB PE = 1 | 29 | 19 | 1.70 |
| SV = 4 | | | |
| Complement factor H OS = Bos taurus OX = 9913 | 24 | 19 | 1.70 |
| GN = CFH PE = 1 SV = 3 | | | |
| Xanthine dehydrogenase/oxidase OS = Bos taurus | 18 | 18 | 1.61 |
| OX = 9913 GN = XDH PE = 1 SV = 4 | | | |
| Protein S100-A9 OS = Bos taurus OX = 9913 | 34 | 16 | 1.43 |
| GN = S100A9 PE = 1 SV = 3 | | | |
| Glycosylation-dependent cell adhesion molecule 1 | 22 | 15 | 1.34 |
| OS = Bos taurus OX = 9913 GN = GLYCAM1 PE = 1 | | | |
| SV = 2 | | | |
| Ribonuclease pancreatic OS = Bos taurus OX = 9913 | 55 | 13 | 1.16 |
| GN = RNASE1 PE = 1 SV = 1 | | | |
| Lipopolysaccharide-binding protein OS = Bos taurus | 26 | 13 | 1.16 |
| OX = 9913 GN = LBP PE = 2 SV = 1 | | | |
| Protein S100-A12 OS = Bos taurus OX = 9913 | 53 | 10 | 0.89 |
| GN = S100A12 PE = 1 SV = 3 | | | |
| Fibroblast growth factor-binding protein 1 OS = Bos | 23 | 10 | 0.89 |
| taurus OX-9913 GN = FGFBP1 PE = 1 SV = 1 | | | |

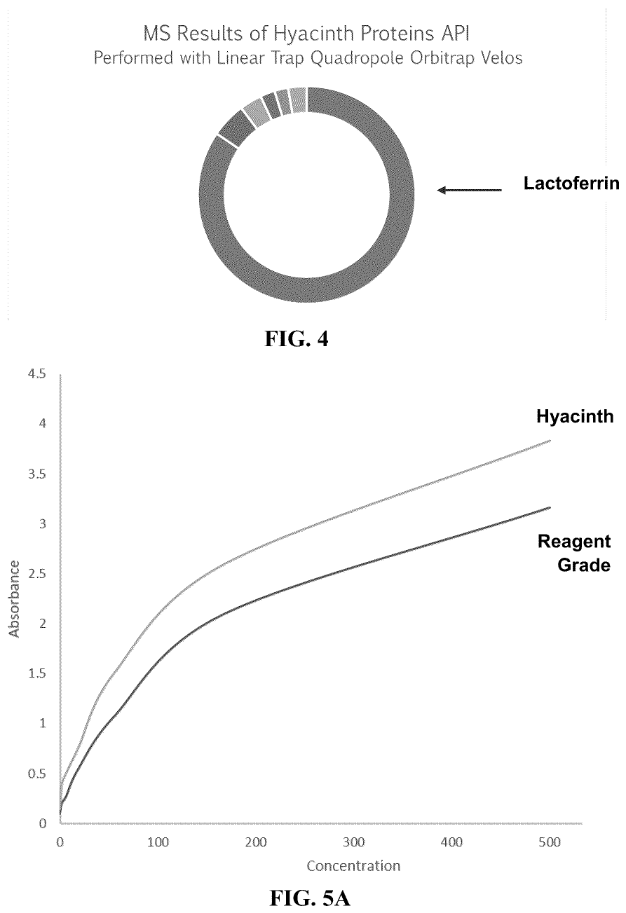

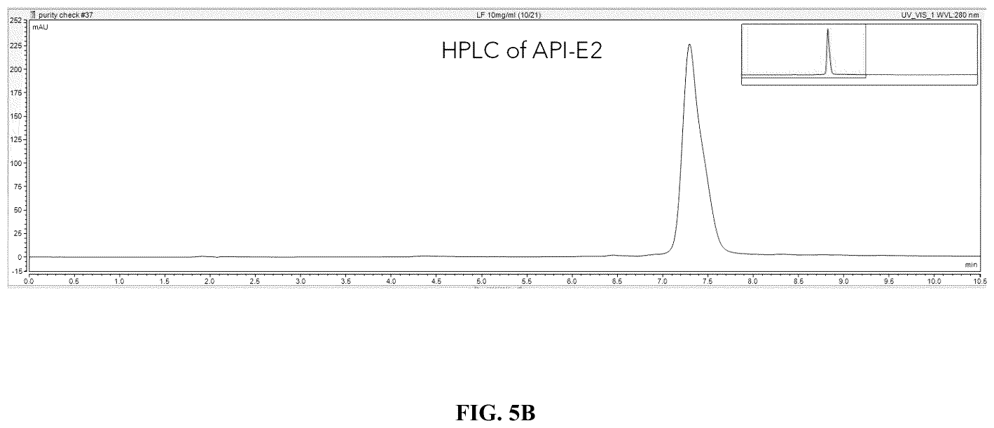

Lactoferrin purity was also assessed by ELISA. As shown in FIG. 5A, when loading the same amount of protein by weight, Lactoferrin produced according to Example 1 (“Hyacinth”) demonstrated a 30% increase in antibody binding relative to existing research standards. Accordingly, the data demonstrated the Hyacinth Proteins purified protein (API-E2) achieved greater purity and/or retained a greater fraction of the native conformational protein state relative to other research grade reference products.

Lactoferrin purity was assessed by HPLC using a Thermo Fisher U3000protein purification system along with a Tricorn 5/150 column packed with Cytiva BigBeads using an NaCl gradient. As shown in FIG. 5B, API-E2 processed by HPLC demonstrates a single peak corresponding to Lactoferrin, indicating ˜100% purity.

The data demonstrate the purification process of Example 1 achieved greater purity of Lactoferrin relative to existing available reagents, particularly in its ability to reduce contamination by Lactoperoxidase.

### Example 3. Assessment of Lactoferrin Activity by Extra-Cellular Matrix Association

Developing an infection requires a multistep process of viral progression. First, a virus targets cells once it enters a host by binding to host Heparan Sulfate (HS) proteoglycans in the Extra-Cellular Matrix (ECM), facilitating the viral particle's binding to its specific receptor on the cell surface. Subsequently, the virus is internalized and replicates inside the host cell. While antiviral drugs typically focus either on inhibiting key viral replication proteins or the specific receptors on the cell surface, the non-specific cellular targeting mechanism of HS in the ECM binding can also be inhibited, interfering with or preventing the virus from binding to target cells. As this pathway is relatively non-specific and requires coating the ECM of exposed cells, traditional therapeutics that take advantage of this approach typically must be dosed at relatively high local concentrations to be maximally effective and generally suffer from impurities and/or toxicity. Accordingly, Lactoferrin preparations with greater purity (e.g., at pharmaceutical grade standards) would offer significantly reduced dosing. In addition, preparation in a manner to retain native function (e.g., retain native conformation, post-translational modifications, iron-binding capacity, etc.) would retain the efficacy of Lactoferrin relative to its activity in raw milk. These preparations (e.g., as prepared in Example 1) allow Lactoferrin to be used, stored, formulated and/or delivered in a greater capacity than other Lactoferrin products.

To assess bioactivity of Lactoferrin, particularly for its potential as an antiviral, binding to the ECM was assessed. Purified Lactoferrin was produced according to Example 1. Caco-2 cells, a human-derived cell culture, cells were allowed to grow for three days after plating on a coverslip such that the cells could proliferate, and the ECM could fully develop. Purified Lactoferrin was then added to the cells at varying concentrations at 37° C. for two hours to allow it to bind as theorized. To directly observe the localization of the API we used Immunofluorescent (IF) imaging and stained both the API and E-cadherin, a cell membrane marker.

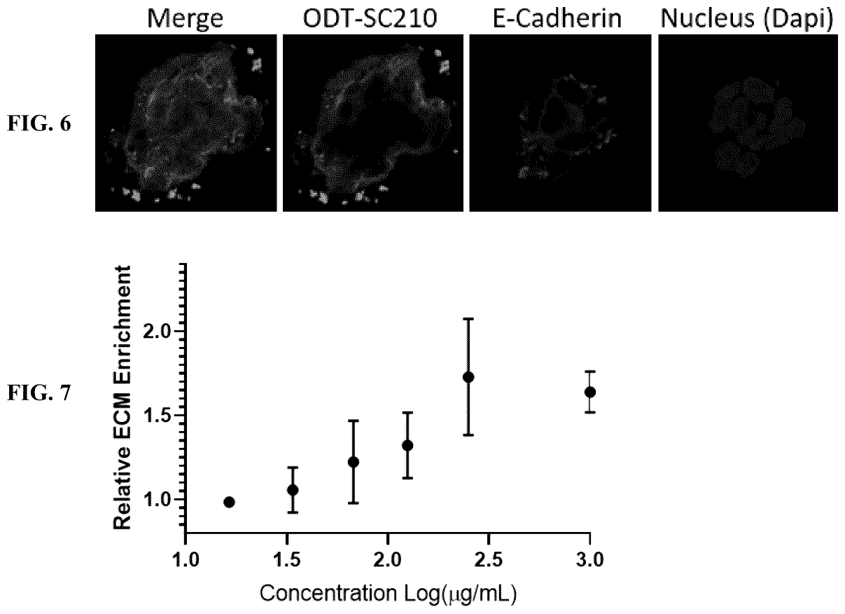

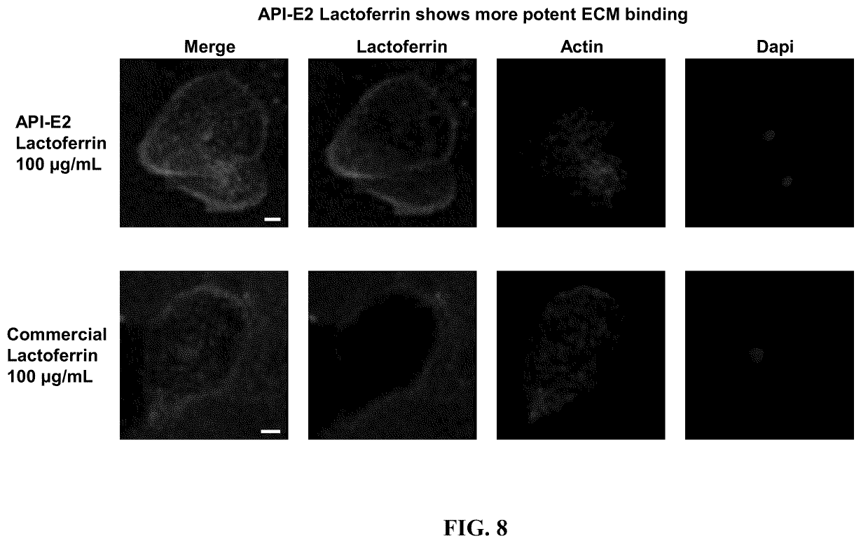

As shown in FIG. 6, purified Lactoferrin enrichment at the ECM was observed as concentration increased. Primary localization internally was also observed at low concentrations, but given Lactoferrin has known intracellular roles and a receptor allowing for its uptake, fractional localization of Lactoferrin to the intracellular space was expected. To quantify the localization of the API, the absolute intensity of the fluorescent signal both just outside/along the E-cadherin mark as well as inside the cell was measured. As shown in FIG. 7, quantification of Lactoferrin localization demonstrated enrichment at the ECM as concentration increased. The results indicate that purified Lactoferrin exhibited the bioactivity of binding to the ECM.

Primary buccal cells were next cultured according to a standard protocol (e.g., see Russo et al. Cytotechnology. 2016 October; 68(5): 2105-2114; herein incorporated by reference for all purposes). For both API-E2 lactoferrin and a commercially available lab-grade standard, lactoferrin was added in media at a final concentration of 100 ug/mL for 1 hr at 37 C, approximately at the concentration where it had been previously found that API-E2 lactoferrin begins to saturate the binding of cultured cells. Immunofluorescence imaging was performed as previously described. As shown in FIG. 8, API-E2 lactoferrin showed strong binding along the ECM of the buccal cells at these conditions (top row). However, the commercially available lab-grade standard showed only limited ECM association of Lactoferrin under the same conditions (bottom row). The results strongly suggest that the API-E2 Lactoferrin is more potent and bioactive in regards to the key host cell ECM association, which is necessary for both the antiviral activity of the protein and biofilm mitigating antibiotic activity.

### Example 4. SARS-COV-2 Antiviral Activity

Cytopathic Effect (CPE) reduction assays were performed to assess the antiviral activity of purified Lactoferrin against SARS-COV-2.

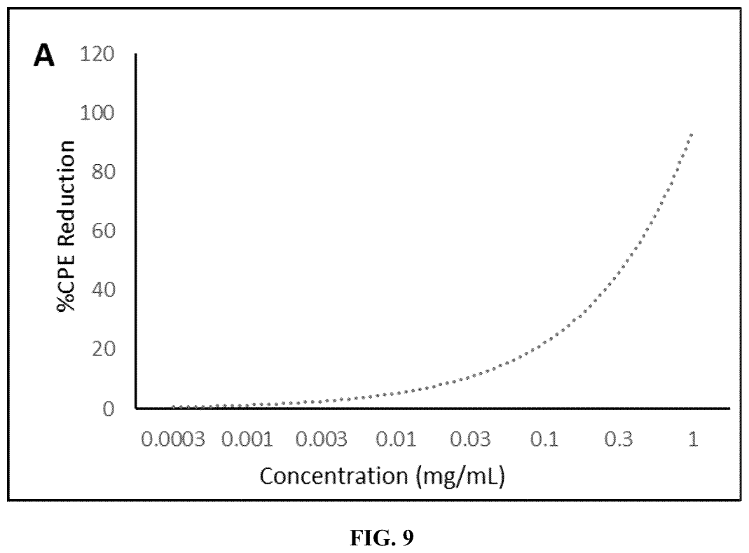

Vero E6 cells were seeded in 96-well cell culture plates at a density of 80-100% confluent cells. Cells were incubated with 3-fold serial dilutions of SC210 starting from a concentration of 1 mg/mL for 2 hr at 37° C. Subsequently, cells were either mock-infected (analysis of cytotoxicity of the compound) or were infected with an MOI of 001 in a total volume of 150 μl of medium with purified Lactoferrin produced according to Example 1, reagent grade Lactoferrin, or Remdesivir. Cell viability was assessed three days post-infection by staining the cells with neutral red dye for two hours, extracting the dye for 30 mins in 50:50 Sorensen citrate buffer/ethanol, and reading out the optical density at OD 540 nm to determine the EC50 (50% effective antiviral concentration). As shown in FIG. 9 and quantified in Table 4, purified Lactoferrin inhibited viral growth, including at a lower concentration than that of comparable research grade reference product. The results demonstrate that purified Lactoferrin produced according to Example 1 inhibited SARS-COV-2 viral infection better than reagent grade suggesting enhanced bioactivity and/or purity.

TABLE 4
EC50 for SARS-CoV-2

| Compound | EC50 (μM) |
|---|---|
| Lactoferrin | 0.09 |
| (“Hyacinth”) | |
| Lactoferrin | 0.14 |
| (“Reagent”) | |
| Remdesivir | 4.5 |

### Example 5. Assessment of Lactoferrin Bioactivity by Differential Scanning Calorimetry

Lactoferrin possesses multiple key bioactivities relevant to its antimicrobial activities, including the important activities of iron chelation and host cell binding. Natively, lactoferrin possesses both iron bound (hololactoferrin) and iron free forms (apolactoferrin). Given lactoferrin binds strongly to iron, without wishing to be bound by theory, hololactoferrin should be substantially more stable than apolactoferrin.

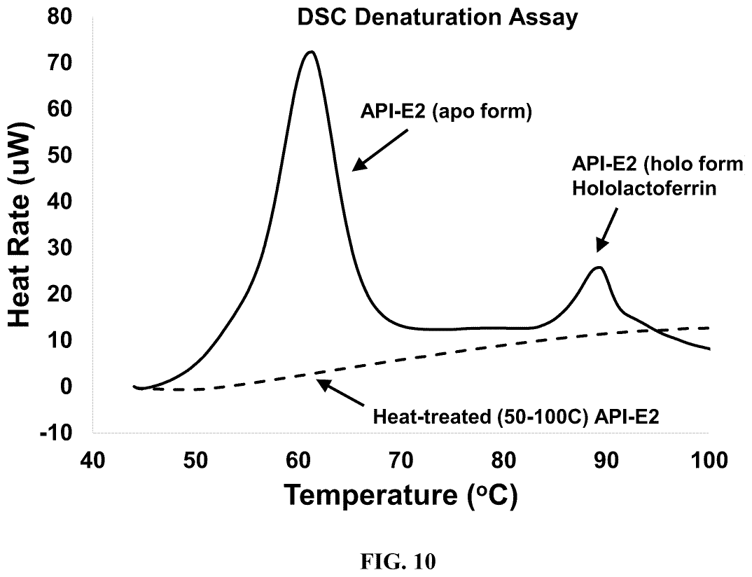

Differential Scanning Calorimetry (DSC) was used to assess the hololactoferrin and apolactoferrin forms through monitoring unfolding temperatures of lactoferrin samples using a nanoDSC (TA Instruments, Lindon, UT) according to standard DSC protocols known to those of skill in the art (e.g., see Hinz et al “MEASUREMENT AND ANALYSIS OF RESULTS OBTAINED ON BIOLOGICAL SUBSTANCES WITH DIFFERENTIAL SCANNING CALORIMETRY”; herein incorporated by reference for all purposes. API-E2 Lactoferrin (10 mg/mL), produced as described herein, was assessed by DSC. As shown in FIG. 10 (solid line), API-E2 Lactoferrin demonstrated two peaks corresponding, from the left, to Apolactoferrin, as the larger peak and Hololactoferrin, as a smaller peak. The lack of other peaks indicates the lack of contaminants and impurities and can assess API-E2 as ˜100%. The results indicate that the presence of iron stabilized Lactoferrin resulting in the shift to higher denaturation temperature in the DSC assay. Accordingly, the DSC assay demonstrated the ability to distinguish Apolactoferrin and Hololactoferrin forms in a given sample. Additionally, the lack of other peaks also indicated the purity of API-E2 Lactoferrin. The API-E2 apolactoferrin peak had a Tm of 60.2+/−0.8 C and a delta H of 466.1 +/−9.2 KJ/mol. The API-E2 hololactoferrin peak has a Tm of 88.38+/−0.8 C and a delta H of 632.7+/−7.2 KJ/mol. The hololactoferrin natively accounted for on average 8.74+/−1.39% of the total API-E2 lactoferrin with apolactoferrin accounting for the rest in the samples tested. However, the hololactoferrin percentage can be naturally variable. The API-E2 was calculated by this technique to contain virtually 100% pure lactoferrin.

TABLE 5
Quantification of Iron Binding Properties from DSC

| | Apolactoferrin | Hololactoferrin |
|---|---|---|
| Tm | 60.2 +/− .8 C. | 88.38 +/− .8 C. |
| Delta H | 466.1 +/− 9.2 kJ/mol | 632.7 +/− 7.2 kJ/mol |
| % of Total | 91.26 +/− 1.39% | 8.74 +/− 1.39% |

The assay was also performed with heat-treated API-E2 Lactoferrin. API-E2 Lactoferrin was heated across a range of 50-100° C. Notably, the range of heat used in the DSC assay is comparable to temperatures typically used in the industry prior to the purification of Lactoferrin (e.g., greater than 63° C.). As shown in FIG. 10 (dashed line), heat-treated API-E2 Lactoferrin demonstrated a loss in both detectable peaks, indicating that both Apolactoferrin and Hololactoferrin forms were fully denatured after the heat treatment.

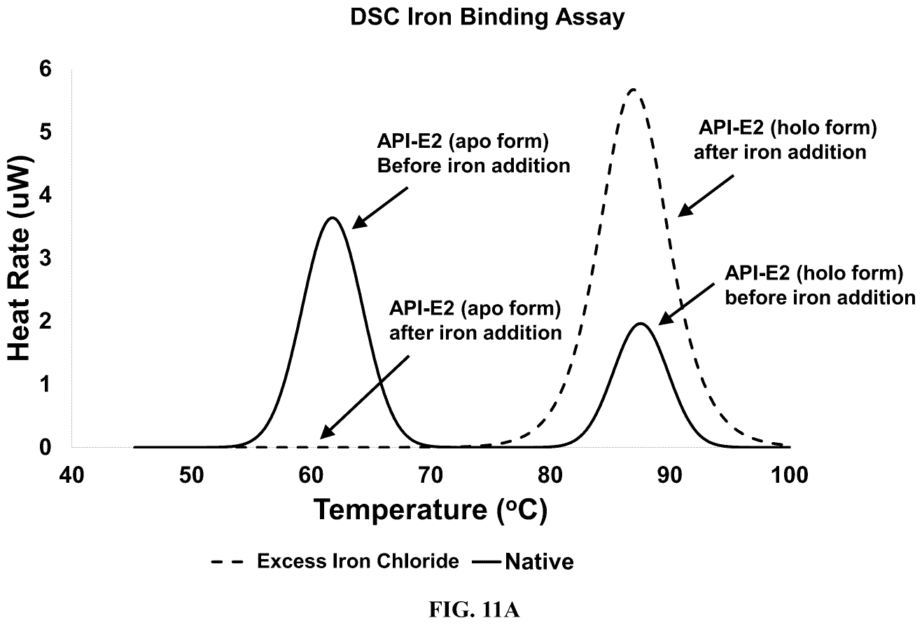

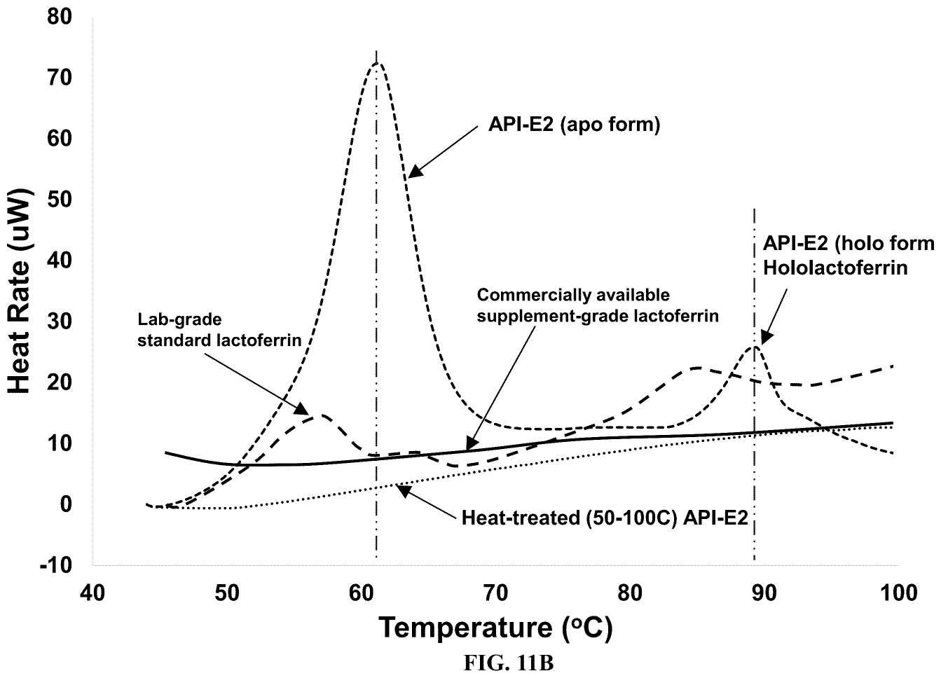

The DSC assay was further used to assess Lactoferrin bioactivity, specifically the iron binding capability of the API-E2 Lactoferrin. Iron chloride (FeC13) was added in 1000× molar excess to API-E2 Lactoferrin. In the presence of excess iron, the API-E2 lactoferrin will bind the iron to its open iron binding site. If the API-E2 has retained its bioactivity through manufacture, all of the Apolactoferrin is expected to shift to the more stable Hololactoferrin form, as determined by the presence of their corresponding DSC peaks. As shown in FIG. 11A, API-E2 Lactoferrin demonstrated two peaks corresponding, on the left, to Apolactoferrin, as the larger peak, and on the right, Hololactoferrin, as the smaller peak in the absence of excess iron. Upon addition of excess iron, 100% of the available Apolactoferrin shifted to the Hololactoferrin form, as demonstrated by the single peak (right peak) of lactoferrin now observed with no detectable Apolactoferrin peak. The results indicate that the iron-binding bioactivity is completely retained in the API-E2 Lactoferrin produced using the methods described herein.

Lab-grade standard lactoferrin and a commercially available supplement-grade lactoferrin were also assessed by DSC under the conditions used to assess API-E2 above. As shown in FIG. 11B, the peaks of the Lab-grade standard lactoferrin were significantly smaller than those observed for API-E2 and less defined, as well as containing 1 extra peak which is attributed to an impurity. The curve possessed a nonspecific upslope as is typically observed in a product with a high degree of denatured protein present in the sample. The estimated ratio of the apolactoferrin to hololactoferrin peak is nearly equal in this result, which indicates a resulting loss of apolactoferrin during processing. As well, both apolactoferrin and hololactoferrin peaks denatured at a lower temperature, 55 and 85 C respectively, which demonstrates a higher degree of instability due to prior chemical, enzymatic and/or heat treatment and have lost iron binding capabilities. Also as shown in FIG. 11B, the commercially available supplement-grade lactoferrin (bottom line) demonstrated no detectable peaks indicating that the product was completely denatured or lacked the presence of the lactoferrin molecule.

### Example 6. Assessment of Lactoferrin Bioactivity on Salivary Bacteria by pH Testing

Salivary bacteria (including Lactobacillus, Lactococcus, and Streptococcus mutans) produce acid during growth. This acidification is strongly associated with tooth decay, particularly when it results in pH below 5.5, which is a clinically relevant threshold. A slurry of these bacteria were treated with sucrose to stimulate growth, as well as varying concentrations of API-E2 lactoferrin. Upon the addition of sucrose and API-E2 lactoferrin, the pH was adjusted to 7 through a titration and the change in pH was subsequently monitored over the next 6 hours.

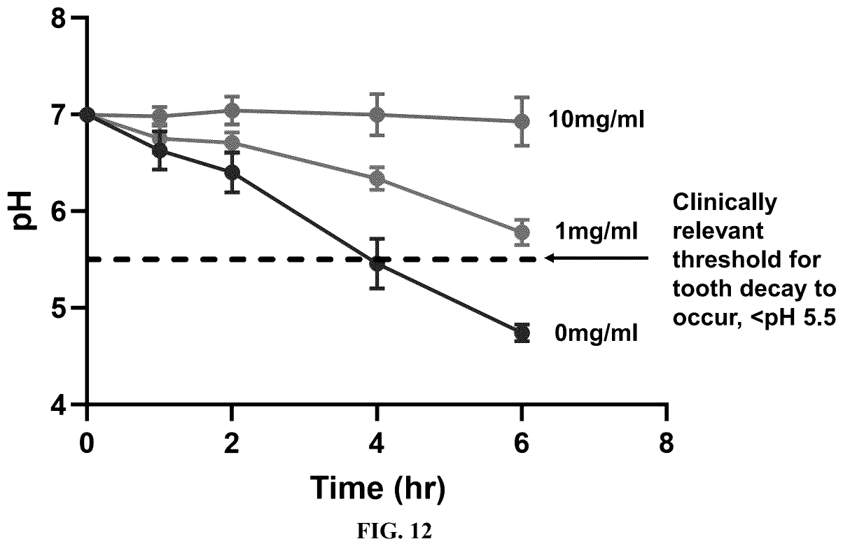

As shown in FIG. 12, without API-E2 Lactoferrin treatment and in the presence of sucrose, salivary bacteria produced acid resulting in pH decreasing to below 5.5 within 4 hours. However, with either 1 mg/mL or 10 mg/mL API-E2 Lactoferrin, in the presence of sucrose, pH is maintained at above 5.5 for the entire 6 hour testing period. The results indicate the presence of API-E2 decreased bacterial acid production in a concentration dependent manner, indicating the antimicrobial bioactivity of the API-E2 Lactoferrin.

### Example 7. Assessment of Lactoferrin Bioactivity by Poultry Surface Treatment

Chicken breasts were inoculated with salivary human bacteria in a slurry as described above in pH testing (including Lactobacillus, Lactococcus, Streptococcus mutans, and Porphyromonas gingivalis) and left for 6 days at 37° C.

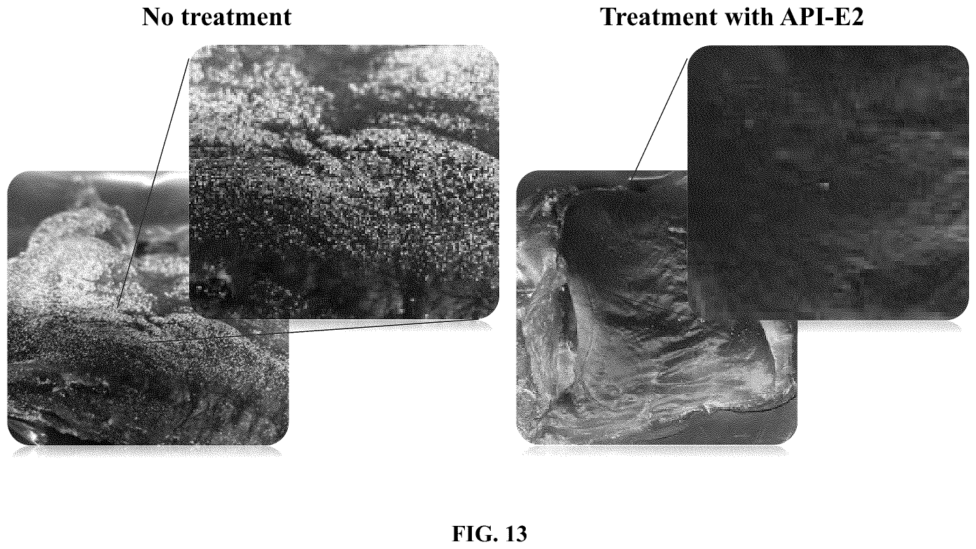

As shown in FIG. 13, without treatment, the chicken breast was coated with bacterial growth at the end of the 6 day window. However, with treatment with 3 mg/mL API-E2 Lactoferrin, no detectable growth of any bacteria colonies was observable after 6 days, indicating antimicrobial bioactivity of the API-E2 Lactoferrin after surface treatment.

### Example 8. Assessment of Lactoferrin Bioactivity by Zone of Inhibition

A zone of inhibition test was performed to assess bioactivity of API-E2 Lactoferrin. A plate with E. coli was cultured with 100 μg/mL, 10 μg/mL, 1 μg/mL, and 0.1 μg/mL API-E2 Lactoferrin added to paper circles and allowed to diffuse out.

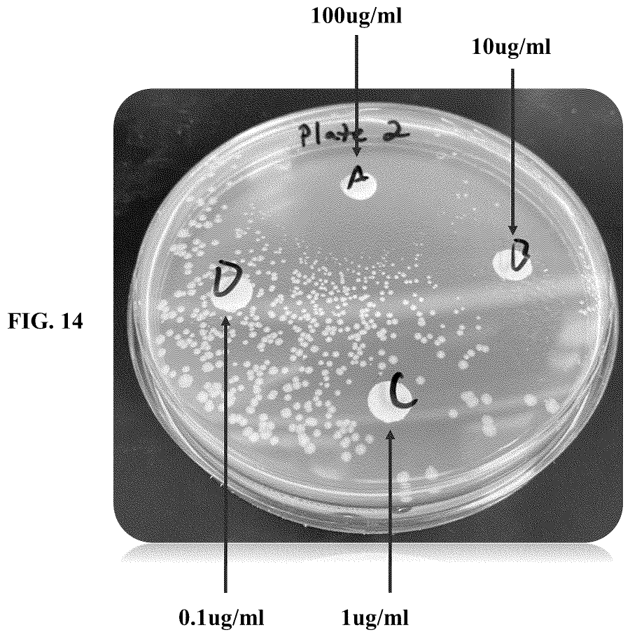

As shown in FIG. 14, complete inhibition was found in the zone of diffusion at 100 μg/mL and 10 ug/mL API-E2 Lactoferrin, together with a limited zone of inhibition at 1 μg/mL, indicating antimicrobial bioactivity of the API-E2 Lactoferrin. No zone of inhibition was seen at 0.1 μg/mL.

### Example 9. Assessment of Lactoferrin Bioactivity by LPO Activity

An enzymatic assay for lactoperoxidase (LPO) activity was developed utilizing a molecule which in the presence of peroxidase activity turns a blue color.

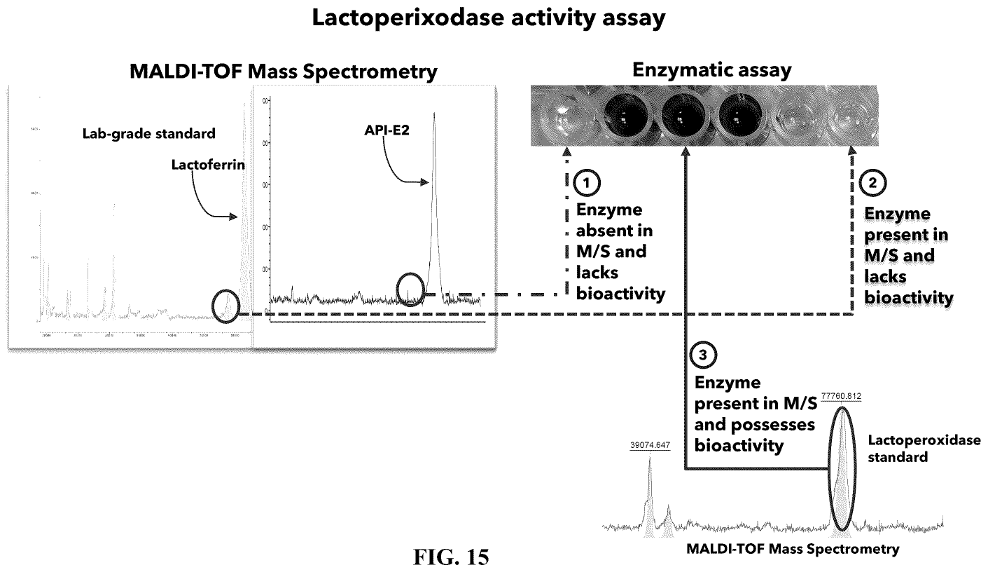

As shown in FIG. 15, for purified LPO standard, which was validated for presence by M/S, this gives a strong positive response (FIG. 15 #3). For API-E2 Lf purified as described herein, LPO was neither detected by M/S or by the enzymatic assay (FIG. 15 #1). However, for the commercially available Sigma lab-grade standard (L9507), we found that contaminant LPO can be seen by M/S, but does not result in a positive enzymatic reaction (FIG. 15 #2). This indicates that Lactoferrin produced by previous methodologies resulted in global loss of bioactivity for proteins treated with these conditions. In the process described herein, however, wherever LPO can be detected by M/S, corresponding enzymatic activity was also observed. Only in samples were LPO is completely or near completely removed from lactoferrin (no M/S is detectable), no enzymatic activity was observed. This indicates both that the API-E2 Lactoferrin product is achieving a very high degree of purity and that the purification process described herein is broadly retaining protein activity throughout purification steps.

### 8. EQUIVALENTS

While the invention has been particularly shown and described with reference to a preferred embodiment and various alternate embodiments, it will be understood by persons skilled in the relevant art that various changes in form and details can be made therein without departing from the spirit and scope of the invention.

### 9. INCORPORATION BY REFERENCE

All references, issued patents and patent applications cited within the body of the instant specification are hereby incorporated by reference in their entirety, for all purposes.

1. The pharmaceutical composition of claim 23, wherein the pharmaceutical composition is formulated for delivery to an oral cavity or nasal cavity.

2. The pharmaceutical composition of claim 1, wherein the pharmaceutical composition is formulated for retention of the Lactoferrin in the buccal mucosa and/or for delivery to the extra-cellular matrix (ECM) of a cell.

3. (canceled)

4. The pharmaceutical composition of claim 1, wherein the pharmaceutical composition is formulated as (i) a tablet, optionally wherein the tablet is formulated as an orally dissolvable or disintegrating tablet; (ii) a spray; (iii) a gum; (iv) a film; or (v) a lozenge.

5. (canceled)

6. (canceled)

7. (canceled)

8. (canceled)

9. (canceled)

10. The pharmaceutical composition of claim 1, wherein the oral cavity or nasal cavity comprises the pharynx.

11. The pharmaceutical composition of claim 23, wherein the pharmaceutical composition is formulated for delivery to a wound.

12. The pharmaceutical composition of claim 11, wherein the pharmaceutical composition is formulated for delivery to skin, optic, connective, muscosa, gastrointestinal and/or lung tissue.

13. The pharmaceutical composition of claim 11, wherein the wound comprises an open wound or ulcer, optionally wherein the ulcer is venous or arterial.

14. (canceled)

15. The pharmaceutical composition of claim 11, wherein the wound is selected from the group consisting of: a burn, a bite, a cut, an infection, an ulceration, and combinations thereof, optionally wherein (i) wherein the burn is selected from the group consisting of: a thermal burn, a chemical burn, an electrical burn, a radiation burn, a friction burn, and combinations thereof; and (ii) wherein the cut is selected from the group consisting of: a puncture, a laceration, an abrasion, an incision, an amputation, and combinations thereof.

16. (canceled)

17. (canceled)

18. The pharmaceutical composition of 11, wherein the pharmaceutical composition comprises a bio-adhesive.

19. The pharmaceutical composition of claim 11, wherein the pharmaceutical composition is formulated in a delivery format selected from the group consisting of: a gelfoam, a hydrogel, an infused bandage, a powder, a spray, and an inhalation format.

20. The pharmaceutical composition of claim 23, wherein the pharmaceutical composition is formulated for delivery to a gastrointestinal tract.

21. The pharmaceutical composition of claim 20, wherein the pharmaceutical composition is formulated in a delivery format comprising microencapsulation and/or wherein the pharmaceutical composition is formulated in a delivery format comprising an enteric coating.

22. (canceled)

23. A pharmaceutical composition comprising Lactoferrin, wherein the composition is formulated for delivery to an oral cavity, nasal cavity, wound, occular cavity, and/or gastrointestinal tract.

24. The pharmaceutical composition of claim 23, wherein the pharmaceutical composition or composition comprises:
(i) an excipient;
(ii) sodium steryl fumate, carbomer, sodium hydroxide, glycerin, sodium benzoate, benzoic acid, citric acid, and/or combinations thereof;
(iii) xylitol, sorbitol, mannitol, maltitol, sugar alcohols, sucrolose, and/or combinations thereof;
(iv) Isomalt, microcrystalline cellulose, Sodium Carboxymethylcellulose, and/or combinations thereof;
(v) comprises powdered sugar, gum base, corn syrup, gum Arabic, and/or combinations thereof;
(vi) comprises HiG PWD-04, Encapsulated powdered flavoring, Anhydrous or Natural Caffeine, Encapsulated Sucralose or Sucralose, Encapsulated Acesulfame-K and Aspartame, Aspartame, Acesulfame-K, Triacetin, Silicon dioxide, and/or combinations thereof;
(vii) a coagulant, optionally wherein the coagulant comprises thrombin, amylopectin, kaolin, and/or combinations thereof; and/or
(viii) an astringent; optionally wherein the astringent is selected from the group consisting of: alum, acacia, sage, yarrow, witch hazel, bayberry, distilled vinegar, persimmon, green tea, black tea, citric acid, cranberry juice and/or extract, grapefruit juice and/or extract, inorganic acids, and combinations thereof.

25. (canceled)

26. (canceled)

27. (canceled)

28. (canceled)

29. (canceled)

30. (canceled)

31. (canceled)

32. (canceled)

33. (canceled)

34. The pharmaceutical composition of claim 23, wherein the Lactoferrin is bovine.

35. The pharmaceutical composition of claim 23, wherein the Lactoferrin has not been treated.

36. The pharmaceutical composition of claim 23, wherein the Lactoferrin has not been chemically treated, enzymatically treated, acid treated, and/or heat treated.

37. (canceled)

38. The pharmaceutical composition or composition of claim 35, wherein the Lactoferrin has not been heat treated at a temperature of 50° C. or greater, 51° C. or greater, 52° C. or greater, 53° C. or greater, 54° C. or greater, or 55° C. or greater.

39. (canceled)

40. The pharmaceutical composition of claim 23, wherein the purified Lactoferrin comprises a native conformation as assessed by (i) circular dichroism and/or (ii) Differential Scanning Calorimetry (DSC), optionally wherein the native conformation comprises an apolactoferrin and/or an hololactoferrin conformation, and optionally wherein the apolactoferrin conformation has a melting temperature peak of 60.2+/−0.8° C. and/or the hololactoferrin conformation has a melting temperature peak of 88.38+/−0.8° C.

41. (canceled)

42. (canceled)

43. (canceled)

44. The pharmaceutical composition of claim 23, wherein the purified Lactoferrin is capable of binding iron, wherein at least 30%, 40%, 50%, 60%, 70%, 80%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, or 99.9% of the purified Lactoferrin is capable of binding iron, optionally wherein the capability to bind iron is assessed by DSC.

45. (canceled)

46. (canceled)

47. (canceled)

48. (canceled)

49. The pharmaceutical composition of claim 23, wherein the purified Lactoferrin comprises a post-translational modification.

50. The pharmaceutical composition of claim 49, wherein the post-translational modification comprises glycosylation.

51. The pharmaceutical composition of claim 23, wherein the purified Lactoferrin comprises an average molecular weight of at least 79000-86000 Da.

52. The pharmaceutical composition of claim 23, wherein the purified Lactoferrin is dried, optionally wherein the purified Lactoferrin is dried by freeze-drying/lyophilization, fluid-bed drying, or low-temperature spray-drying.

53. (canceled)

54. The pharmaceutical composition of claim 23, wherein the purified Lactoferrin remains in liquid form through Lactoferrin purification.

55. The pharmaceutical composition of claim 23, wherein the composition further comprises an iron molecule, optionally wherein the iron molecule comprises Fe²⁺ or Fe³⁺.

56. The pharmaceutical composition of claim 23, wherein the purified Lactoferrin is complexed with an iron molecule, optionally wherein the iron molecule comprises Fe²⁺ or Fe³⁺.

57. (canceled)

58. The pharmaceutical composition of claim 23, wherein the purified Lactoferrin is complexed with a copper, zinc, manganese, and/or gallium molecule.

59. (canceled)

60. The pharmaceutical composition of claim 23, wherein the composition comprises endotoxin at a level of 5 EU/kg or less.

61. The pharmaceutical composition of claim 23, wherein the Lactoferrin has been purified from a natural milk product that has not been treated.

62. The pharmaceutical composition of claim 61, wherein the natural milk product has not been processed prior to purification of the Lactoferrin.

63. The pharmaceutical composition of claim 61, wherein the natural milk product has not been chemically, enzymatically, acid, or heat treated prior to purification of the Lactoferrin.

64. (canceled)

65. The pharmaceutical composition of claim 63, wherein the natural milk product has not been heat treated prior to purification of the Lactoferrin at a temperature of 50° C. or greater, 51° C. or greater, 52° C. or greater, 53° C. or greater, 54° C. or greater, or 55° C. or greater.

66. (canceled)

67. The pharmaceutical composition of claim 23, wherein the Lactoferrin has been purified from a natural milk product that has been separated into skim milk and cream prior to purification of the Lactoferrin, optionally wherein the separation into skim milk and cream comprises cold-bowl separation.

68. (canceled)

69. The pharmaceutical composition of claim 23, wherein the Lactoferrin has been purified from a natural milk product has been acid treated prior to purification of the Lactoferrin, optionally wherein the acid treatment comprises removal of insoluble caseins.

70. (canceled)

71. The pharmaceutical composition or composition of claim 69, wherein the acid treatment is at a pH of 4.0 or greater.

72. A method of manufacturing the pharmaceutical composition of claim 23.

73. The method of claim 72, wherein the method comprises one or more steps selected from the group consisting of: chromatography, filtration, and drying, optionally wherein the method comprises each of the steps of chromatography, filtration, and drying.

74. (canceled)

75. The method of 72, wherein the method comprises pressing into a dosage form.

76. A method of treating a disease, optionally a pathogenic disease, or condition, the method comprising administering the pharmaceutical composition of claim 23.

77. The method of claim 76, wherein:
(a) the disease or condition is selected from the group consisting of: a pathogenic disease, a gastrointestinal (GI) infection, a wound infection, an allergic condition, an inflammatory condition, and combinations thereof; and/or
(b) the administration is prophylactic; and/or
(c) the subject (i) is at risk for exposure to a pathogen; (ii) has been exposed to a pathogen; or (iii) has been diagnosed with an infection by a pathogen; and/or
(d) the pathogen comprises:
(i) an orally and/or nasally transmitted microbe;
(ii) an airborne microbe;
(iii) a virus, optionally wherein the virus comprises a coronavirus; and optionally wherein the coronavirus is SARS-COV-2; and/or
(iv) a bacterium or a fungus.

78. (canceled)

79. (canceled)

80. (canceled)

81. (canceled)

82. (canceled)

83. A method of reducing the risk of a pathogenic disease or the risk of an infection, the method comprising administering the pharmaceutical composition of claim 23 to a subject.

84. The method of claim 83, wherein;
(a) reducing the risk for the pathogenic disease or the infection comprises administration of the pharmaceutical compositions or compositions to a wound of the subject; and/or
(b) the administration is prophylactic; and/or
(c) the subject has been exposed to a pathogen and/or has been diagnosed with an infection by a pathogen; and/or
(d) the pathogen comprises:
(i) an orally and/or nasally transmitted microbe;
(ii) an airborne microbe;
(iii) a virus, optionally wherein the virus comprises a coronavirus; and optionally wherein the coronavirus is SARS-COV-2; and/or
(iv) a bacterium or a fungus.

85. (canceled)

86. (canceled)

87. (canceled)

88. (canceled)

89. (canceled)

90. (canceled)

91. (canceled)

92. (canceled)

93. (canceled)

94. (canceled)

95. A method of promoting wound healing of a tissue, the method comprising administering the pharmaceutical composition of claim 23, to a wound of a subject's tissue.

96. The method of claim 95, wherein:
(a) the wound is selected from the group consisting of: a burn, a cut, an infection, an ulceration, and combinations thereof, optionally (i) wherein the burn is selected from the group consisting of:
wherein the burn is selected from the group consisting of: a thermal burn, a chemical burn, an electrical burn, a radiation burn, a friction burn, and combinations thereof; or (ii) wherein the cut is selected from the group consisting of: a puncture, a laceration, an abrasion, an incision, an amputation, and combinations thereof; and/or
(b) the tissue is skin, lung tissue, or combinations thereof; and/or
(c) the method comprises treating a disease or condition selected from the group consisting of: a pathogenic disease, a gastrointestinal (GI) infection, a wound infection, an allergic condition, an inflammatory condition, and combinations thereof, optionally wherein treating the disease or condition comprises a prophylactic treatment of the disease or condition.

97. (canceled)

98. (canceled)

99. (canceled)

100. (canceled)

101. (canceled)
